(19) United States
(12) Patent Application Publication
(10) Pub. No.: US 2022/0362430 A1
(43) Pub. Date: Nov. 17, 2022

(54) VOLATILE MATERIAL DISPENSER

(71) Applicant: S. C. Johnson & Son, Inc., Racine, WI (US)

(72) Inventors: Sebastian D. Hasik, Antioch, IL (US); Gregory G. Pieper, Spring Grove, IL (US); Katherine Cusatis, Wauwatosa, WI (US); James T. Walker, Racine, WI (US); Brian T. Davis, Burlington, WI (US); Timothy R. Ordiway, Racine, WI (US); Timothy Grala, Antioch, IL (US); Kamran Faterioun, New Berlin, WI (US); Douglas A. Soller, Racine, WI (US)

(21) Appl. No.: 17/878,629

(22) Filed: Aug. 1, 2022

Related U.S. Application Data
(63) Continuation-in-part of application No. 16/579,489, filed on Sep. 23, 2019, now Pat. No. 11,407,000.

(51) Int. Cl. A61L 9/12

(52) U.S. Cl. CPC A61L 9/122; A61L 9/127; A61L 2209/133; A61L 2209/134; A61L 2209/132

(57) ABSTRACT

A volatile material dispenser includes a base and a stand assembly that is coupled with the base. The base includes a fan and a battery therein. The stand assembly includes a platform, a stand that extends from the platform, and a manifold that extends from the stand. The volatile material dispenser also includes a shroud and an air deflector. The shroud is positioned on the base and defines a chimney. The air deflector is positioned between the shroud and the manifold. The air deflector is configured to direct air out of the chimney.

### CROSS REFERENCE TO RELATED APPLICATIONS

This application is a continuation-in-part of U.S. application Ser. No. 16/579,489, filed on Sep. 23, 2019, which is hereby incorporated by reference herein in its entirety.

### BACKGROUND OF THE DISCLOSURE

### 1. Field of the Disclosure

The present disclosure relates generally to volatile material dispensers for emitting volatile materials and, more particularly, to volatile material dispensers having a piezoelectric element and lighting assembly.

### 2. Description of the Background of the Disclosure

Various volatile material dispensers are known in the art, most of which deliver fragrance to the surrounding environment by a variety of different mechanisms. For example, some dispensers spray a volatile containing a fragrance into the surrounding environment, while other dispensers allow for the evaporation of a volatile containing a fragrance into the surrounding environment. Such volatile material dispensers generally include a housing with a refill inserted therein. The refill generally includes a container for holding a volatile material, and the volatile material may include various components such as aroma chemicals, water, solvents, surfactants, alcohols, and/or other components. Some refills include a wick in contact with the volatile material and extending out of the refill to carry the volatile material out of the refill. Other refills include a gel-like substance that is emitted through a semi-permeable membrane. Regardless of the type of refill, the refill may be inserted into a volatile material dispenser having a heater, a piezoelectric element, an aerosol actuator, and/or any other diffusion element that may assist in delivering the volatile material.

However, many prior art dispensers do not distribute volatile materials in a uniform or accurately dosed fashion. Further, many dispensers fail to include visual feedback to a user regarding the amount or quantity of volatile that is being or has been dispensed by the volatile material dispenser. As such, there is a need for a volatile material dispenser having a mechanism that accurately disperses a volatile while providing adjustable, visual feedback to a user.

### SUMMARY OF THE INVENTION

According to a first aspect, a volatile material dispenser includes a base and a stand assembly that is coupled with the base. The base includes a fan and a battery therein. The stand assembly includes a platform, a stand that extends from the platform, and a manifold that extends from the stand. The volatile material dispenser also includes a shroud and an air deflector. The shroud is positioned on the base and defines a chimney. The air deflector is positioned between the shroud and the manifold. The air deflector is configured to direct air out of the chimney.

According to some embodiments, the air deflector comprises a frustoconical shape and includes an air wall. In some embodiments, a plurality of air channels are defined by the air wall of the air deflector and a side surface of the manifold. In some embodiments, the air wall extends around the manifold. In some embodiments, the base further includes a plurality of vents. In some embodiments, the base further includes an air isolator that has an intake aperture. In some embodiments, the air isolator is positioned between the fan and the battery. In some embodiments, during use, air is configured to flow into the plurality of vents, through the intake aperture of the air isolator, and out of the chimney.

According to another aspect, a volatile material dispenser includes a base and a stand assembly that is coupled with the base. The base includes a fan and a battery therein. The base defines a lower portion that comprises a plurality of vents. The stand assembly includes a manifold, and the manifold contains a piezoelectric assembly. The volatile material dispenser further comprises a shroud and an air deflector. The shroud defines a chimney, and the air deflector is positioned within the shroud. During use, air is configured to flow through the plurality of vents and out of the chimney.

In some embodiments, the base further comprises an air isolator and a sealed engine. In some embodiments, the sealed engine is waterproof. In some embodiments, the sealed engine is positioned above the fan and underneath a platform of the stand assembly. In some embodiments, a printed circuit board and a plurality of light emitting diodes are positioned within the sealed engine. In some embodiments, the piezoelectric assembly is connected with the printed circuit board. In some embodiments, the fan comprises a motor that is positioned within the sealed engine. In some embodiments, the volatile material dispenser is configured to be used in an outdoor environment.

According to yet another aspect, a volatile material dispenser includes a base and a stand assembly that is coupled with the base. The stand assembly includes a manifold. The manifold contains a piezoelectric assembly. The volatile material dispenser also comprises a fan and a refill that has a wick. The refill is removably coupled with the manifold and comprises a volatile material therein. The volatile material dispenser further comprises a shroud positioned around the manifold and the refill. The shroud defines a chimney. The manifold includes an annular wall. The annular wall defines a passageway. The annular wall is coaxial with the chimney and the piezoelectric assembly. During use, a plume of volatile is configured to flow through the passageway and the chimney by way of the piezoelectric assembly.

In some embodiments, the wick of the refill is in contact with the piezoelectric assembly when the refill is coupled with the manifold. In some embodiments, the volatile material dispenser further comprises an air deflector that is secured with the shroud and the manifold. In some embodiments, the air deflector is configured to direct air out of the chimney and around the plume of volatile. In some embodiments, the base further includes a plurality of vents and an air isolator.

### BRIEF DESCRIPTION OF THE DRAWINGS

FIG. 1 is a front, top, and right isometric view of a volatile material dispenser in accordance with the present disclosure;

FIG. 2 is a front elevational view of the volatile material dispenser of FIG. 1;

FIG. 3 is a rear elevational view of the volatile material dispenser of FIG. 1;

FIG. 4 is a left side elevational view of the volatile material dispenser of FIG. 1;

FIG. 5 is a top plan view of the volatile material dispenser of FIG. 1;

FIG. 6 is a bottom plan view of the volatile material dispenser of FIG. 1;

FIG. 7 is a front, top, and right isometric view of an internal stand and a base of the volatile material dispenser of FIG. 1 with a shroud having been removed for clarity;

FIG. 8 is a left side elevational view of the stand and base of FIG. 7;

FIG. 9 is a front elevational view of the stand and base of FIG. 7;

FIG. 10 is a front, top, and right isometric view of a refill for use with the volatile material dispenser of FIG. 1;

FIG. 11 is a front, top, and right isometric view of the refill of FIG. 10 with a cap having been removed;

FIG. 12 is a side cross-sectional view of the refill taken through line 12-12 of FIG. 11;

FIG. 13 is a cross-sectional view of the volatile material dispenser taken through line 13-13 of FIG. 5;

FIG. 14 is a cross-sectional view of the volatile material dispenser taken through line 14-14 of FIG. 5;

FIG. 15 is a front, top, and left isometric view of various electrical components of the volatile material dispenser of FIG. 1, including a piezoelectric element;

FIG. 16 is a top plan view of a printed circuit board and some of the electrical components shown in FIG. 15;

FIG. 17 is a front, top, and right isometric view of the piezoelectric element shown in FIG. 16;

FIG. 18A is a cross-sectional view of the piezoelectric element taken through line 18-18 of FIG. 17;

FIG. 18B is a schematic illustration of a circular-shaped array of apertures along the piezoelectric element of FIG. 17;

FIG. 18C is a schematic illustration of an oval-shaped array of apertures along the piezoelectric element of FIG. 17;

FIG. 19 is a detail view of an upper portion of the internal stand of FIG. 14;

FIG. 20 is a detail view of the upper portion of the internal stand of FIG. 19 with the refill of FIG. 10 inserted into a manifold of the stand;

FIG. 21 is a line graph illustrating the results of various tests of springs having varying thicknesses;

FIG. 22 is a line graph illustrating the results of a piezoelectric element and various tests comparing spring force against a resultant fragrance output;

FIG. 23 is a front, right, and top view of the stand and base of FIG. 7 with the refill of FIG. 10 being inserted into a head cavity thereof;

FIG. 24 is a front, right, and top view of the stand and base of FIG. 23 with the refill having been inserted into an engaged configuration;

FIG. 25 is a front, right, and top view of the stand and base of FIG. 23 with a shroud being engaged with the base;

FIG. 26 is a top, front, and left isometric view of a volatile material dispenser, according to another embodiment of the present disclosure;

FIG. 27 is a front elevational view of the volatile material dispenser of FIG. 26;

FIG. 28 is a rear elevational view of the volatile material dispenser of FIG. 26;

FIG. 29 is a right side elevational view of the volatile material dispenser of FIG. 26;

FIG. 30 is a top plan view of the volatile material dispenser of FIG. 26;

FIG. 31 is a bottom plan view of the volatile material dispenser of FIG. 26;

FIG. 32 is a front, top, and right isometric view of an internal stand assembly and a base of the volatile material dispenser of FIG. 26 with a shroud having been removed for clarity;

FIG. 33 is a left side elevational view of the stand and base of FIG. 32;

FIG. 34 is a front elevational view of the stand and base of FIG. 32;

FIG. 35 is a partial, enlarged, front, top, and right isometric view of the stand of FIG. 32 with a sidewall and a lower portion of the base having been removed for clarity;

FIG. 36 is a cross-sectional view of the volatile material dispenser taken through line 36-36 of FIG. 30;

FIG. 37 is a cross-sectional view of the volatile material dispenser taken through line 37-37 of FIG. 30;

FIG. 38 is a zoomed in isometric cross sectional view of the base of the volatile material dispenser taken through line 38-38 of FIG. 30;

FIG. 39 is a front, top, and left isometric view of a refill for use with the volatile material dispenser of FIG. 26;

FIG. 40 is a front, top, and right isometric view of various electrical components of the volatile material dispenser of FIG. 26, including a piezoelectric element; and

FIG. 41 is a detail view of an upper portion of the volatile material dispenser of FIG. 26 with the refill of FIG. 39 inserted into a manifold of the stand.

### DETAILED DESCRIPTION

The present disclosure is directed to volatile material dispensers or diffusers and methods of emitting volatile materials therefrom. While the present disclosure may be embodied in many different forms, several specific embodiments are discussed herein with the understanding that the present disclosure is to be considered only an exemplification of the principles of the disclosure, and it is not intended to limit the disclosure to the embodiments illustrated. Throughout the disclosure, the terms “about” and “approximately” refer to a range of values ±5% of the numeric value that each term precedes. As noted herein, all ranges disclosed within this application are inclusive of the outer bounds of the range.

The volatile material dispensers or diffusers disclosed herein are multisensory devices that use a piezoelectric engine to create and eject micro droplets of liquid fragrance into the surrounding environment. The volatile material dispensers are configured to accept a replaceable fragrance oil bottle or refill. The dispensers are further configured to run on low voltage, and feature a fragrance intensity selector that provides visual feedback to a user in the form of a glowing light. The dispenser includes a shroud, a stand assembly, and a base that collectively house control buttons, a power connector, lights, and a piezo engine. The shroud is primarily decorative and provides a light transmission shade. Before a first use of the volatile material dispenser, the shroud is removed to insert a fragrance oil bottle or refill into the stand assembly.

Still further, the volatile material dispensers disclosed herein include improvements to a piezo plate-to-wick interface, which, through testing, has been found to increase system performance and consistency. Specifically, testing has revealed a sensitivity and variations in consistency based upon a downward contact force of the piezo plate and a top of the wick. For example, a constant force provides a more consistent output and minimized variance. Through testing, it has also been determined that force differences can create variances in output rate as a result of changing load conditions on the piezo plate. Too high of a downward load onto the piezo plate has been found to dampen an amplitude of mechanical vibration of the piezo plate, which can minimize droplet output. Conversely, too light of a downward force has been found to limit the plate-to-wick interface contact, which has been found to result in high, undamped output. This is likely because under such circumstances, contact between the piezo plate and the wick can be lost entirely.

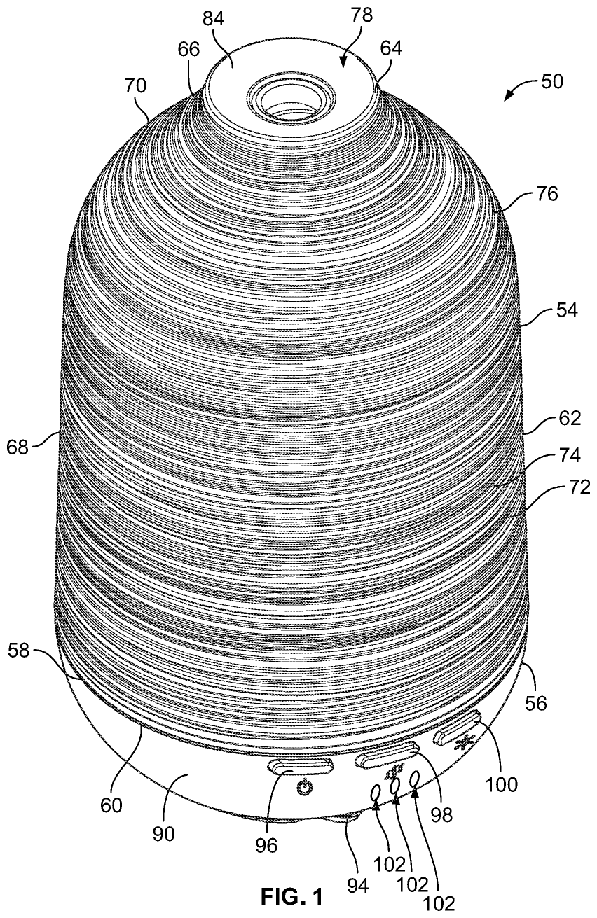

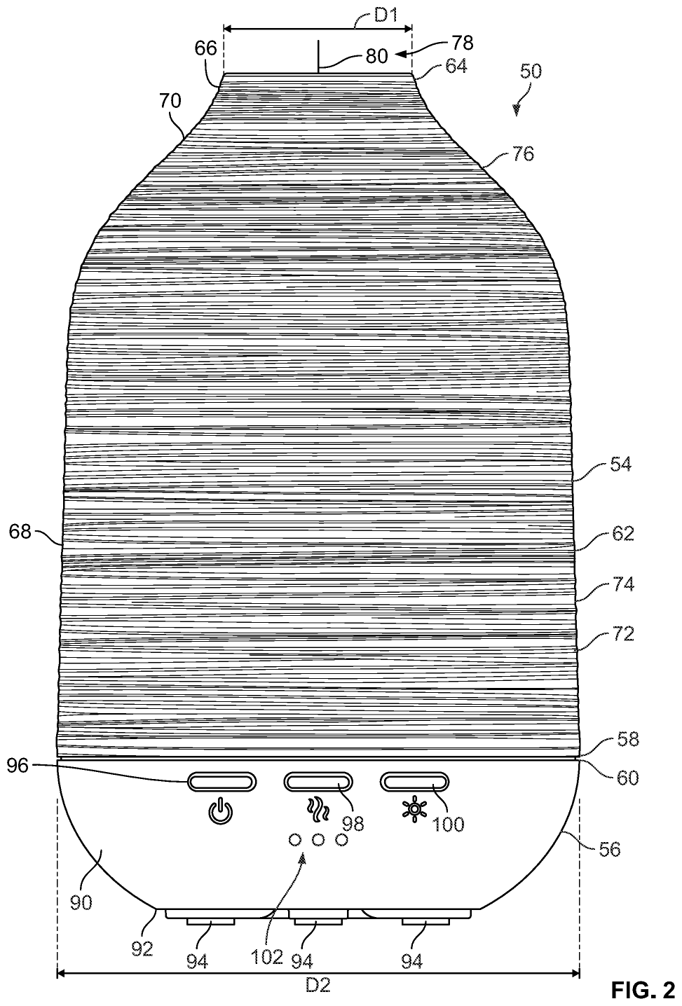

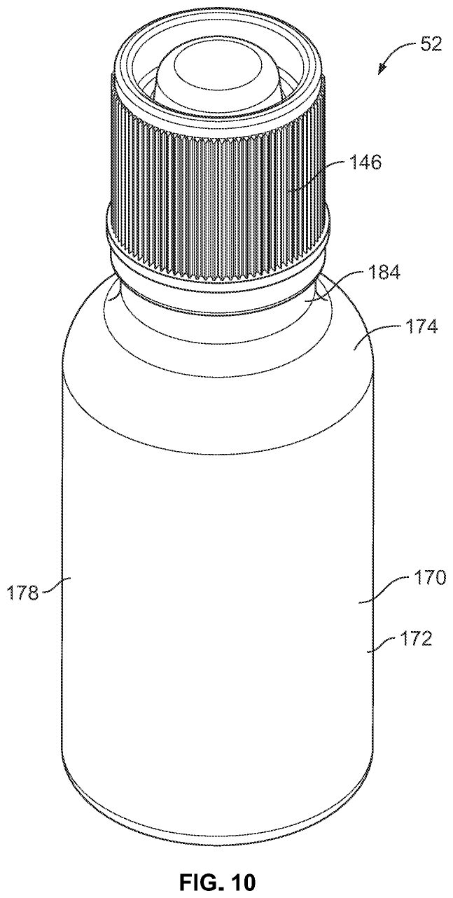

Referring now to the drawings, FIGS. 1-7 depict a volatile material dispenser 50 embodying aspects of the present disclosure. The dispenser 50 may be adapted to accommodate a refill 52 (see FIGS. 10-12) and dispense a volatile material in the form of water and/or fragranced oil from the refill 52. Referring specifically to FIGS. 1 and 2, the dispenser 50 includes a shroud or cover 54 and a base 56. The shroud 54 defines a lower edge 58 that interfaces with an upper edge 60 of the base 56. A sidewall 62 of the shroud 54 extends upward from the lower edge 58 toward a lip 64 at an upper end 66 of the shroud 54. The sidewall 62 of the shroud 54 defines a frustoconical lower portion 68 and a spline-shaped upper portion 70, when viewed in cross-section. A plurality of ridges or design features 72 are provided along an outer surface 74 of the shroud 54. Any number of design features 72 may be provided along the outer surface 74 of the shroud 54. The lower portion 68 and the upper portion 70 of the shroud 54 intersect at a rounded shoulder 76, from which the upper portion 70 extends toward a chimney 78 that is centered about a central longitudinal axis 80 (FIG. 2) of the dispenser 50. The chimney 78 is defined as the portion interior to the lip 64. In some embodiments, the shroud 54, and the base 56 comprise polypropylene (PP), however, the shroud 54 and the base 56 may comprise a wide variety of polymeric materials.

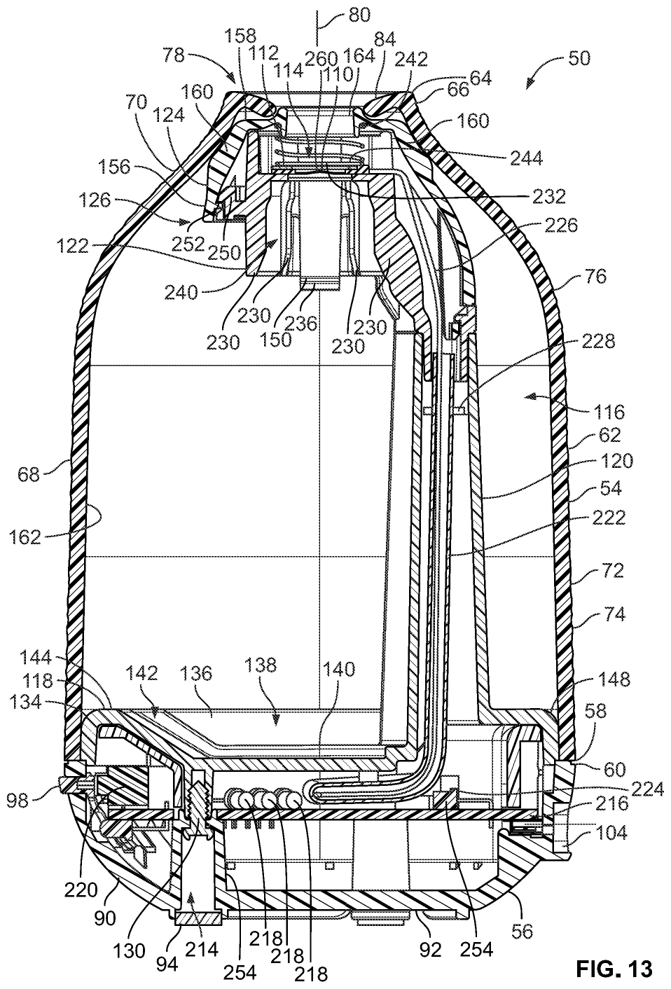

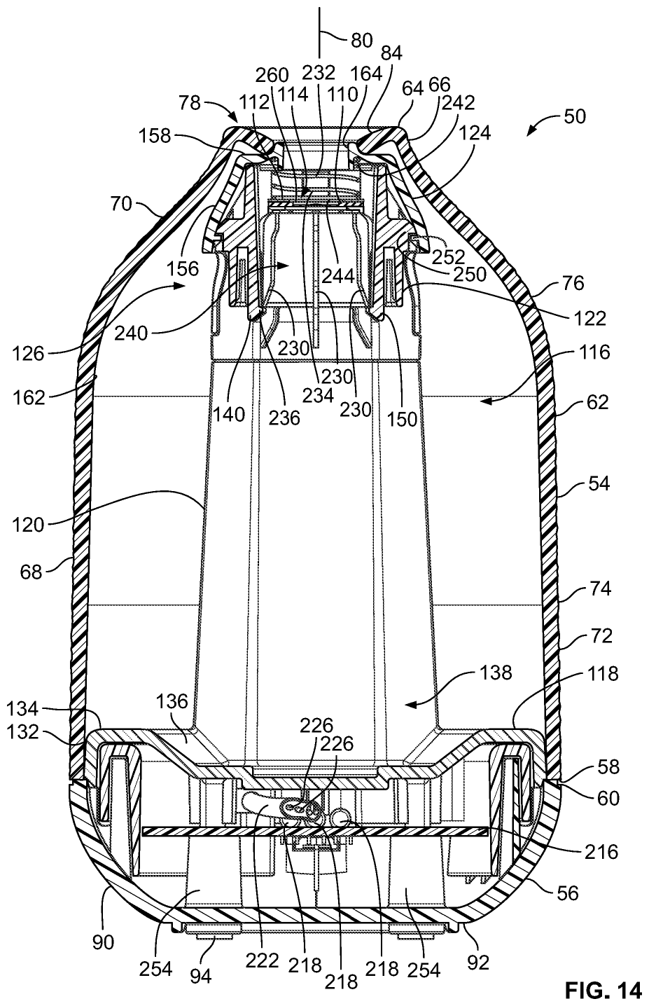

Still referring to FIGS. 1 and 2, the sidewall 62 along the upper portion 70 extends from the shoulder 76 toward the lip 64. Referring to FIG. 1, a flange 84 of the shroud 54 extends downward from the lip 64 toward the longitudinal axis 80 and into the chimney 78. The angle at which the flange 84 extends downward from the lip 64 is best illustrated in the cross-sectional views of FIGS. 13 and 14. Referring specifically to FIG. 2, while the chimney 78 may define a variety of diameters, the chimney 78 is illustrated having a diameter D1 that is about 30% of a widest diameter D2 of the shroud 54 taken at the lower edge 58 thereof. In some embodiments, the diameter D1 may be between about 5% and about 60% of the diameter D2, or between about 10% and about 45% of the diameter D2, or between about 15% and about 35% of the diameter D2. In some embodiments, the diameter D1 is about 10%, or about 20%, or about 30%, or about 40%, or about 50% of the diameter D2.

Referring again to FIGS. 1 and 2, the base 56 also defines a sidewall 90 that extends from the upper edge 60 of the base 56 downward, toward a bottom wall 92 thereof. A plurality of feet 94 extend downward from the bottom wall 92, and are provided to allow the dispenser 50 to rest upon a flat surface (not shown). The sidewall 90 of the base 56 is generally curved, and extends from the upper edge 60 downward and inward, toward the longitudinal axis 80. The sidewall 90 of the base 56 may define a radius of curvature along a portion of the sidewall 90. Still further, a first button 96, a second button 98, and a third button 100 extend from the base 56 outward, away from the longitudinal axis 80. The first button 96, the second button 98, and the third button 100 may be used for a variety of purposes, and may have a variety of different functions, as discussed hereinafter below.

Referring to FIG. 2, the first button 96, the second button 98, and the third button 100 may be provided along the base 56 of the dispenser 50. The first button 96 may be a power button that allows a user to turn the dispenser 50 on and off. The second button 98 may be a fragrance strength adjusting button that allows a user to cycle through settings of the dispenser 50. For example, the user may be able to switch between a low setting, a medium setting, and a high setting. A plurality of light indicators 102 may be visible through the base 56 adjacent or below the second button 98. The light indicators 102 may provide visual feedback to a user regarding the strength or intensity of the chosen setting. In the illustrated embodiment, there are three of the light indicators 102. When the low setting is selected, one of the light indicators 102 may be illuminated; when the medium setting is selected, two of the light indicators 102 may be illuminated; and when the high setting is selected, three of the light indicators 102 may be illuminated. The third button 100 may be a light illumination button that, when pressed by a user, cycles through various light brightness settings and color settings.

A user can select an option for light and/or color based on personal preference. Further, in some embodiments, after being turned on via the first button 96, the dispenser 50 may run for a pre-determined amount of time, e.g., 8 hours, and may subsequently enter into a sleep mode for a pre-determined amount of time, e.g., 16 hours. The dispenser 50 may repeat this cycle every 24 hours unless manually turned off via the first button 96 by a user. In some embodiments, the dispenser 50 includes an automatic shut-off function that deactivates the dispenser 50 after a particular period of time, for example, after seven 24-hour cycles, i.e., one week. In some embodiments, a limit switch (not shown) may be provided along the base 56 which only allows the dispenser 50 to be activated when the shroud 54 is engaged with the base 56.

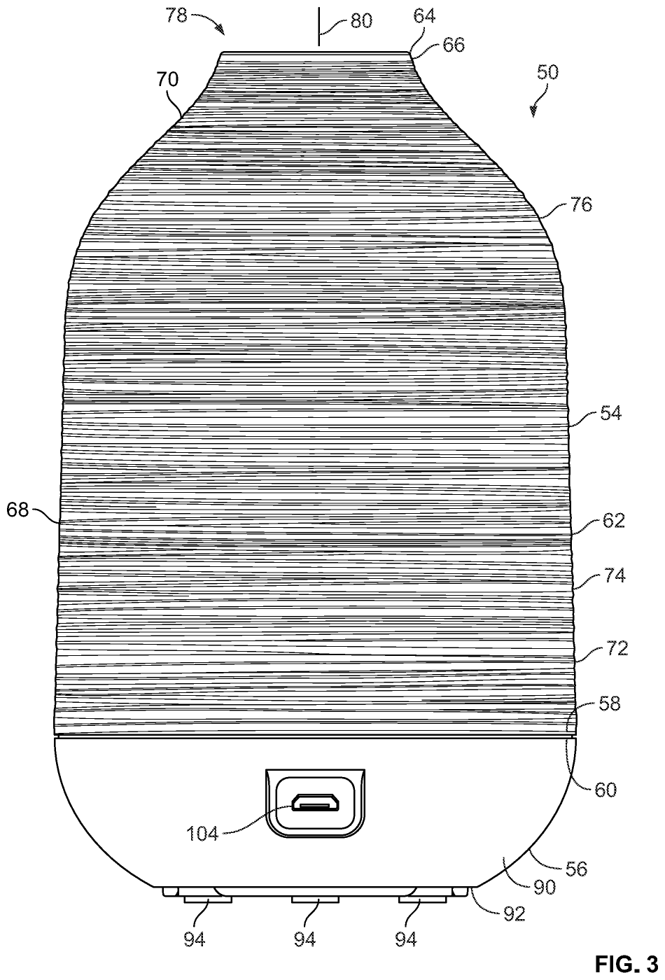

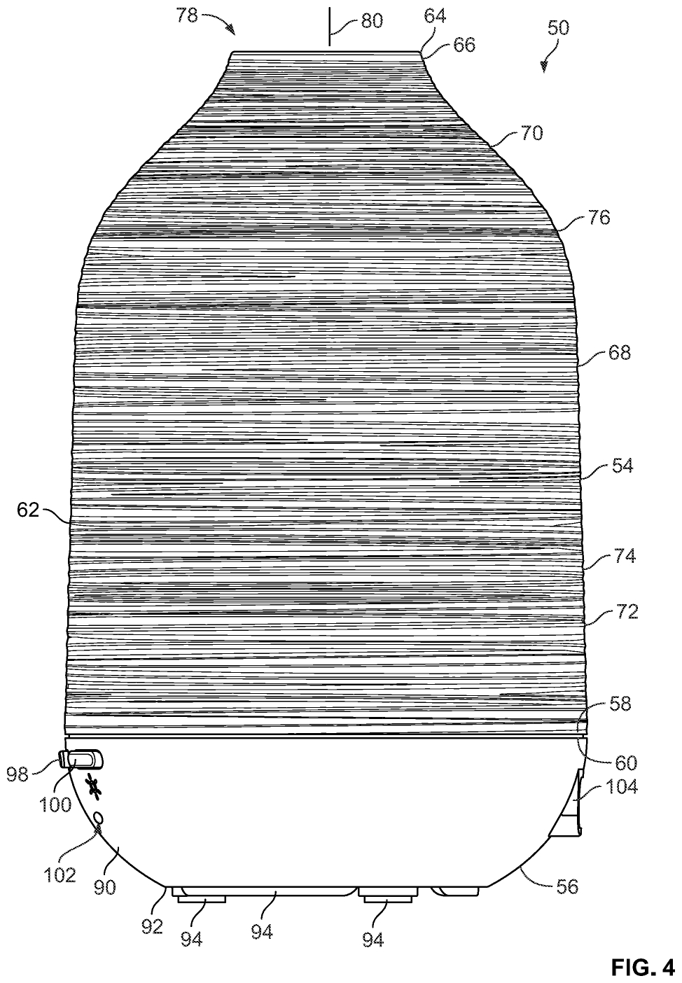

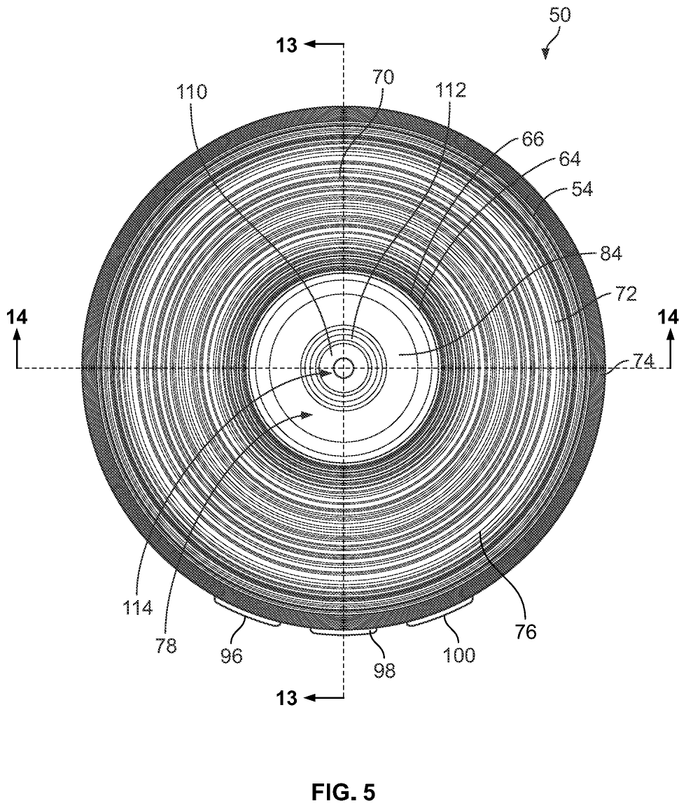

Referring now to FIG. 3, a rear view of the dispenser 50 is shown. As illustrated in the rear view, the dispenser 50 further includes a low voltage receptacle or port 104 for receiving a low-voltage electrical connector of a low voltage wire, such as a USB cord (not shown). In some embodiments, electrical prongs, a cord, or another suitable electrical connector may be electrically coupled with the dispenser 50 so as to allow electrical power to be provided to the dispenser 50. FIG. 4 illustrates a left side of the dispenser 50. The buttons 96, 98, 100 and the port 104 extend outward from the sidewall 90 of the base 56 and cause interruptions of the sidewall 90. As a result, the sidewall 90 of the base is rotationally symmetrical, but the entire base 56 is symmetric about a plane 13-13 (see FIG. 5) that bisects the second button 98 and the port 104. Additional features may be provided within or along the shroud 54 and/or the base 56. Further, additional bodies defining one or more sidewalls (not shown) may be provided between, above, or below the base 56 and the shroud 54.

Referring now to FIG. 5, a top view of the dispenser 50 is depicted. The chimney 78 is shown in greater detail, which is centered along the longitudinal axis 80. A piezoelectric assembly 110 is shown within the chimney 78, which is also centered along the longitudinal axis 80. An annular piezoelectric element 112 is also visible through the chimney 78, the piezoelectric element 112 defining a circular rim of the piezoelectric assembly 110. The piezoelectric element 112 includes a central aperture 114 through which liquid volatile within the refill is dispersed when the dispenser 50 is activated. When the dispenser 50 is activated, a stream of the volatile is dispensed through the chimney 78 of the shroud 54 and out of the dispenser 50, as discussed in greater detail hereinafter below.

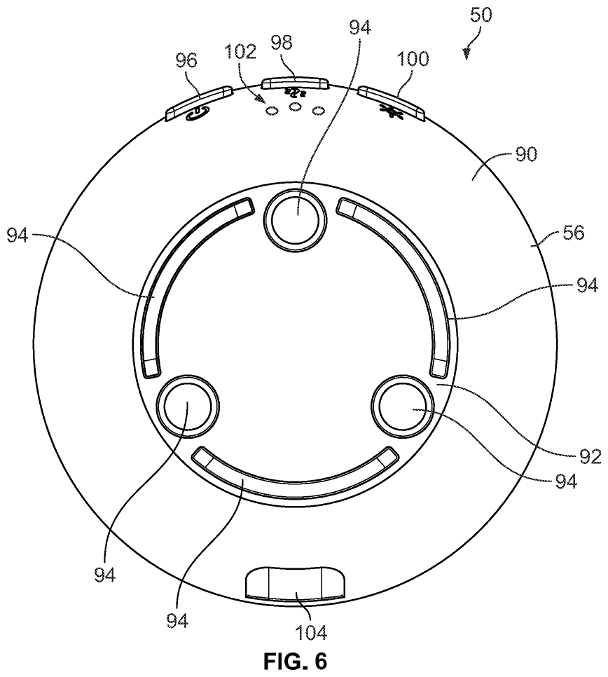

Referring now to FIG. 6, the bottom wall 92 of the base 56 is shown in greater detail. The plurality of feet 94 extend from the bottom wall 92. The plurality of feet 94 are provided in a generally circular shape, with alternating circular and elongate feet 94. Alternative configurations or shapes of the feet 94 are contemplated, while in some embodiments additional structure may be included in addition to the feet 94. Still further, in some embodiments, there are no feet 94 or other types of stability mechanisms that retain the dispenser 50 in an upright configuration. As further illustrated in FIG. 6, the second button 98 and the port 104 are provided 180 degrees offset from one another about the longitudinal axis 80. The first button 96, the second button 98, and the third button 100 are also equally spaced apart from one another. Alternative configurations of the port 104 and the buttons 96, 98, 100 are also contemplated.

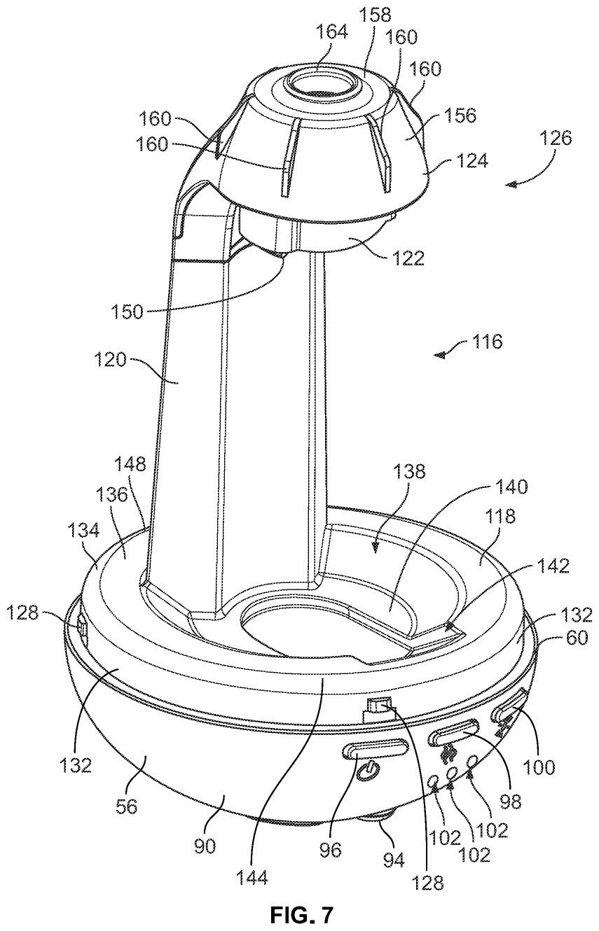

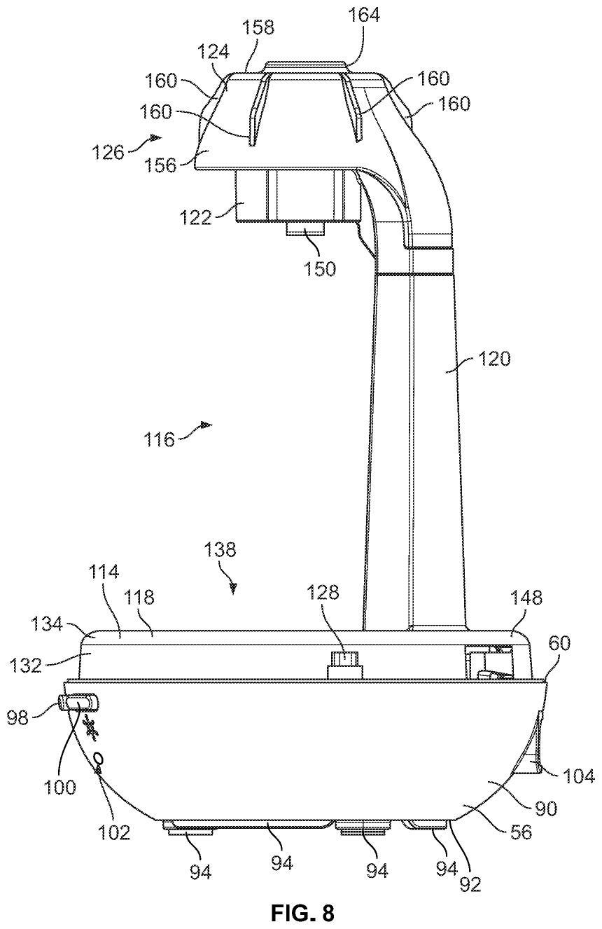

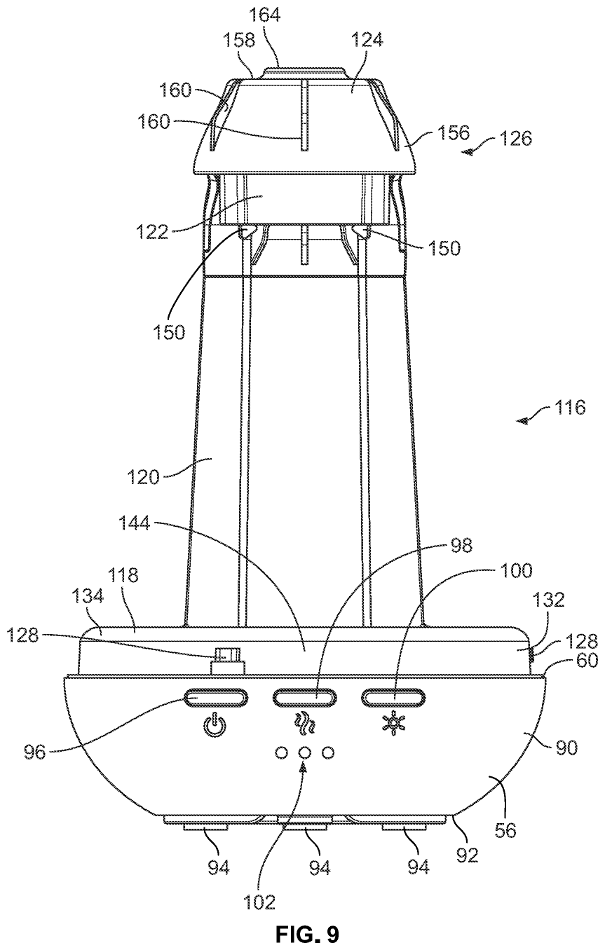

Referring now to FIGS. 7-9, a stand assembly 116 of the dispenser 50 is shown, with the shroud 54 having been removed for clarity. The stand assembly 116 includes a platform 118, a stand 120, a refill chassis 122, and a crown 124. The refill chassis 122 and the crown 124 define a manifold 126 that retains the refill 52 and the piezoelectric assembly 110. The platform 118 extends upward from the base 56 and is inset from the sidewall 90 thereof. A plurality of retention protrusions 128 extend outward from the platform 118, which operate to engage the shroud 54 when it is coupled with the base 56. The retention protrusions 128 are a form of retention mechanism that couples the shroud 54 with the base 56. Alternative retention mechanisms may include elements that cause a snap fit, a friction fit, or an interference fit. The platform 118 is permanently coupled with the base 56 via a plurality of fasteners 130 (see FIG. 13), the channels for which are covered by the feet 94 after assembly thereof. The fasteners 130 are a form of fastening mechanism, additional examples of which include rivets, nails, bolts, or cables.

The platform 118 defines an outer cylindrical surface 132 that extends upward to a corner 134. An angled surface 136 extends inward and downward from the corner 134, toward a well 138 defined within a central portion 140 of the platform 118. A slot 142 is defined within the angled surface 136 along a front portion 144 of the platform 118. The slot 142 may be included to aid in the insertion of the refill 52 into an operable configuration. The well 138 may include additional features that aid in retaining the refill 52 or another component. For example, the well 138 of the platform 118 may be sized and shaped to retain a cap 146 (see FIG. 10) of the refill 52 after the cap 146 has been removed from the refill 52.

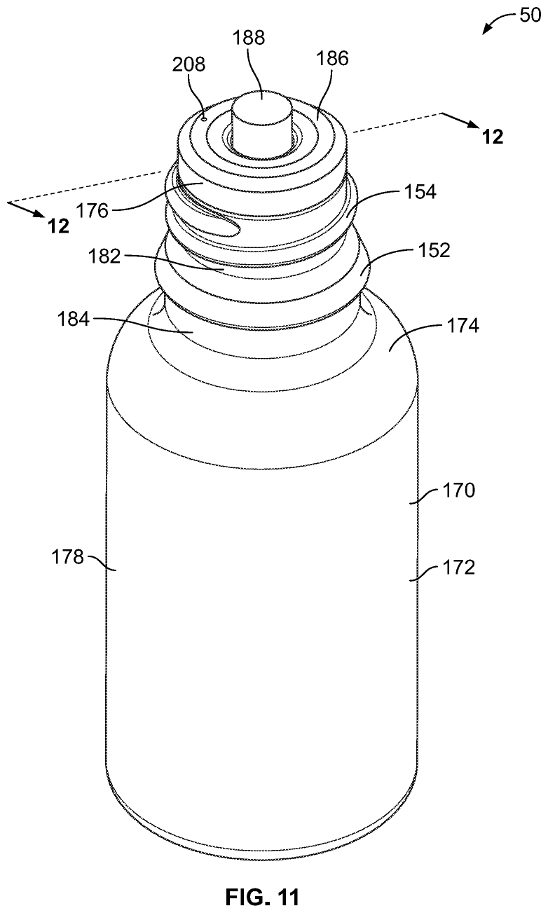

Still referring to FIGS. 7-9, the stand assembly 116 includes the stand 120, which extends upward from the platform 118 along a rear portion 148 thereof. The stand assembly 116 comprises a unitary piece that extends upward from the platform 118 toward the manifold 126, which extends inward, and aligns with the longitudinal axis 80. Elements of the stand assembly 116 may also comprise polypropylene or another type of polymeric material. As noted above, the manifold 126 includes the crown 124 and the refill chassis 122, which extends downward from the crown 124. The chassis 122 includes the first and second tabs 150 that depend downward to retain an annular rim 152 (see FIG. 11) of the refill 52. As will be discussed in greater detail hereinafter below, the first and second tabs 150 may be formed to flex outward when the refill 52 is laterally inserted into the chassis 122 until the rim 152 of the refill engages with the tabs 150.

The refill 52 may be removed from the tabs 150 by a user grabbing the refill 52 and laterally pulling the refill down. In some embodiments, the forces required for insertion and removal of the refill 52 are low enough to simply allow for lateral insertion and removal. However, in some embodiments, the refill 52 may be removed by squeezing the tabs 150 to deflect the tabs 150 outward, thereby releasing the rim 152 from engagement therewith. In some embodiments, the refill 52 may be disengaged by rotating the refill 52 such that threading 154 (see FIG. 11) allows the refill to be rotated and translate downward, away from the crown 124, for removal from the manifold 126. Alternative removal mechanisms may be implemented, such as one or more buttons (not shown) that could be depressed to cause the refill 52 to be released from the chassis 122.

With continued reference to FIGS. 7-9, the crown 124 extends upward from the chassis 122 and defines a generally frusto-conical side surface 156 that terminates at a top surface 158. A plurality of ribs 160 are radially disposed along the side surface 156, which may be formed to aid in physical engagement with an underside 162 (see FIG. 13) of the shroud 54. The ribs 160 are spaced apart about the longitudinal axis 80. A cylindrical wall 164 extends upward from the top surface 158 of the crown 124, the cylindrical wall 164 having a center that is coaxial with the longitudinal axis 80. The cylindrical wall 164 is also coaxial with the chimney 78 defined by the shroud 54. The cylindrical wall 164 is aligned with the second button 98 and the port 104, i.e., the plane 13-13 (see FIG. 5) extends through all of the cylindrical wall 164, the second button 98, and the port 104. While the shroud 54 generally comprises radial symmetry notwithstanding the design features 72, the platform 118, the stand 120, and the manifold 126 may be characterized as being symmetric about the plane 13-13 that intersects the second button 98 and extends through the longitudinal axis 80.

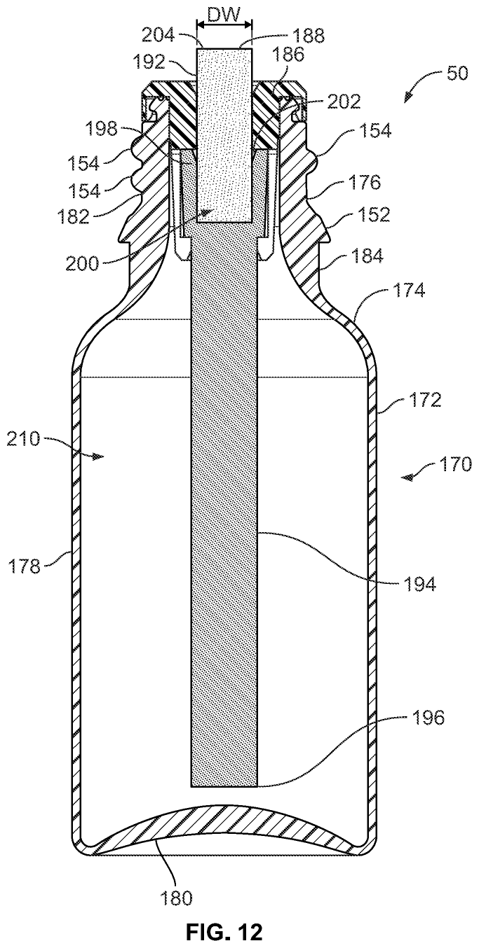

Referring now to FIGS. 10-12, the refill 52 is shown in greater detail. The refill 52 comprises a cap 146 and a container 170 defining a body 172, a shoulder 174, and a head 176. The body 172 comprises a cylindrical outer wall 178 that extends upward from a lower wall 180 thereof to the shoulder 174. The head 176 extends upward from the shoulder 174 and defines a finish 182. The head 176 joins the body 172 at the shoulder 174. The refill 52 further includes a cap 146 that is threadably coupled with the threading 154 (see FIGS. 11 and 12) disposed along a neck 184 of the refill 52. Referring to FIG. 12, the container 170 holds a volatile material therein, and the container 170 is adapted to be retained within the dispenser 50. A wick holder or plug 186 is disposed within the neck 184 for holding a wick 188 with a first end of the wick 188 in contact with the volatile material and a second end of the wick 188 extending out of the container 170 through the neck 184. In illustrative embodiments, the wick 188 may be formed of extruded fibers that are bundled together into the shape of a rod.

Still referring to FIG. 12, the wick 188 may be formed of rope or one or more cotton cords. The wick 188 comprises an upper wick 192 and a lower wick 194. The upper wick 192 has different properties than the lower wick 194, however, in some embodiments, the upper wick 192 and the lower wick 188 have the same properties. In a preferred embodiment, the upper wick 192 is pliable and/or fibrous, while the lower wick 194 may be sintered or more rigid than the upper wick 192. The upper wick 192 may have properties that allow the upper wick 192 to deform into the piezoelectric assembly 110. The lower wick 194 includes a lower end 196 disposed adjacent and spaced apart from the lower wall 180, and has an upper end 198 that is engaged with the upper wick 192. The upper wick 192 is nested within a wick cavity 200 at the upper end 198 of the lower wick 194. An upper cambered or angled surface 202 surrounds an inner periphery of the wick cavity 200. The upper angled surface 202 may aid in alignment of the upper wick 192 within the wick cavity 200 during assembly of the refill 52. The upper wick 192 is snugly retained by the plug 186 and has a distal end 204 that extends upward, out of the container 170. The wick 188 may be formed in any suitable shape or of any suitable material, however, the upper wick 192 is preferably more pliable than the lower wick 194. The plug 186 is retained within the neck 184 of the refill 52 by an interference fit, a friction fit, or in any other suitable manner that holds the plug 186 in place within the neck 184.

Still referring to FIG. 12, the shoulder 174 of the container 170 extends upward to the finish 182 and the neck 184 of the head 176. The threading 154 circumscribes the neck 184 and extends outward therefrom. The annular rim 152 also circumscribes the finish 182, which may be used to engage the refill 52 with the depending tabs 150 of the chassis 122. The lower wall 180 of the refill 52 is generally concave and extends upward, toward the wick 188. An air hole 208 (see FIG. 11) is provided within the plug 186 to allow air to enter into a cavity 210 of the refill 52 as liquid (not shown) is dispersed out of the refill 52 into the surrounding atmosphere. Although a refill 52 is shown and described with particularity, it is contemplated that any type of refill may be used with variations of the dispensers described herein. For example, a refill with a flexible container may be utilized. Still further, the delivery system (i.e., the wick) may be different and/or the size and/or the shape of the container 170 may be different than those described herein.

The volatile material disposed in the container 170 may be any type of volatile material adapted to be dispensed into an environment. For example, the material within the container 170 may include a cleaner, an insecticide, an insect repellant, an insect attractant, a disinfectant, a mold or mildew inhibitor, a fragrance, a disinfectant, an air purifier, an aromatherapy scent, an antiseptic, an odor eliminator, a positive fragrancing volatile material, an air-freshener, a deodorizer, or the like, and combinations thereof. Additives may be included in the volatile material, such as, for example, fragrances and/or preservatives. In fact, any fluid may be provided within the container 170.

Referring now to the cross-sectional views of FIGS. 13 and 14, internal components of the dispenser 50 are shown in greater detail. Referring to FIG. 13, the base 56 is shown being connected with the stand assembly 116 via one of the fasteners 130. The fastener 130 is disposed within a fastener channel 214 that is covered from view by one of the feet 94 after the stand assembly 116 has been fastened to the base 56. In the present embodiment, there are three fasteners 130 that secure the base 56 with the stand assembly 116, however, only a single fastener 130 is shown in the cross-section of FIG. 13.

A printed circuit board (PCB) 216 is shown intermediate the base 56 and the stand assembly 116. A plurality of light emitting diodes (LEDs) 218 are shown electrically coupled with the PCB 216, the plurality of LEDs 218 being disposed above the PCB 216. In some embodiments, the plurality of LEDs 218 are disposed above and below the PCB 216. In some embodiments, some of the LEDs 218 are disposed adjacent the front, rear, and sides of the dispenser 50. As noted above, the LEDs 218 are intended to be used to emit light through the shroud 54 depending on the chosen setting, which can vary based on user preference. The LEDs 218 may alternatively be positioned in any suitable location within the dispenser 50. The one or more LEDs 218 may indicate that the dispenser 50 is on or off, may provide an alert, and/or may provide any other suitable indicator for a user. As noted above, a color and/or a brightness of the LEDs 218 may be adjusted depending on a desired brightness and/or color of light to be emitted through the shroud 54.

For example, a first one of the LEDs 218 may illuminate a first color when the dispenser 50 is in a “Low” setting, a second one of the LEDs 218 may illuminate a second color when the dispenser 50 is in a “Medium” setting, and a third one of the LEDs 218 may illuminate a third color when the dispenser 50 is in a “High” setting. The third LED 218 may illuminate by itself in the high setting, or the lighting may be additive, such that both that first LED 218, the second LED 218, and the third LED 218 illuminate in the high setting, in which they may have the same or different colors and/or intensities. Alternatively, the first LED 218 may be illuminated when the dispenser 50 is plugged in but not on, and the second LED 218 may be illuminated when the dispenser 50 is plugged in and turned on. The dispenser 50 may include one or more separate openings in the shroud 54 or translucent portions of the shroud 54 to permit passage of the light emitted by each LED 218.

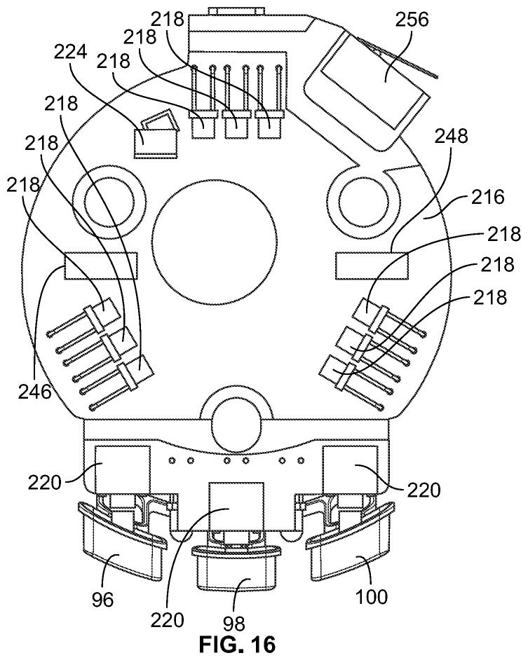

Still referring to FIG. 13, the second button 98 is shown protruding through the base 56. While the second button 98 is shown and referred to hereinafter below, the first button 96 and the third button 100 have an identical setup and functionality. The second button 98 is shown adjacent a switch 220, the switch 220 being operable to be adjusted between various settings of the dispenser 50. The switch 220 may be a push button switch that, when depressed, may cause the dispenser 50 to initiate one or more functions, such as turning the dispenser 50 on or off, causing an adjustment in the amount of fragrance that is dispensed, or adjusting the color or brightness of one or more of the LEDs 218. The switch 220 that is visible in FIG. 13 is one of a plurality of switches 220 that are visible in FIG. 16. The switches 220 may be the same switches, or the switches 220 may be different.

While the dispenser 50 is disclosed as having particular switches, one skilled in the art will appreciate the dispenser may include any number of switches and/or may include any suitable types of switches, for example, timing switches, on/off switches, setting switches, switches controlling another component within the assembly, such as a heater or a fan, and/or any other suitable switches.

Still referring to FIG. 13, the stand assembly 116 is shown in cross section, and the components that are disposed therein are visible. A wire tube 222 is shown, which extends from a terminal 224 that is disposed along the PCB 216, through the stand 120, and into the chassis 122. First and second wires 226 are disposed within the wire tube 222, which are electrically connected to the terminal 224 and to the piezoelectric assembly 110, which is disposed within the crown 124 of the stand assembly 116. The stand 120 is fixedly coupled with the chassis 122, which is also fixedly coupled with the crown 124. The stand 120, the chassis 122, and the crown 124 are all separate components, and may be coupled with one another via a snap fit, friction fit, interference fit, adhesive, or another method of coupling. As noted above, the stand 120, the chassis 122, and the crown 124 may all comprise polypropylene, or another polymeric material. The wires 226 are electrically coupled with the piezoelectric assembly 110, which is disposed within the crown 124. A wire tube guide 228 is further disposed within the stand 120, which is situated to retain the wire tube 222 within the stand 120. The wire tube guide 228 may comprise a horizontal portion having a cutout that retains the wire tube 222 in a static configuration. As further shown in FIG. 13, the piezoelectric assembly 110 is centrally located along the longitudinal axis 80. While the stand 120, the chassis 122, the platform 118, the base 56, and the shroud 54 are illustrated as comprising a polymer, other types of materials are contemplated.

Still referring to FIG. 13, a plurality of refill retaining ribs 230 are disposed along an interior of the chassis 122, which may be used to guide the neck 184 of the refill 52 upward, into position within the chassis 122 until the tabs 150 lock into place with the rim 152 along the refill 52. A spring 232 is shown, which is used to apply a force against the piezoelectric assembly 110. The spring 232 applies a constant force against the piezoelectric assembly 110 to retain it in a static configuration until the refill 52 as it is positioned within the chassis 122 by a user. When a user inserts the refill 52 into the dispenser 50, the piezoelectric assembly 110 is translated upward, causing the spring 232 to compress. The spring 232 defines a spring wire 234 that has a wire diameter, which is further discussed below. One of the tabs 150 is also shown, the tab 150 including an inwardly-depending catch 236 that is configured to translate outward, away from the longitudinal axis 80 as the refill 52 is being inserted into the chassis 122, and snaps back inward to secure the refill 52 in place within the chassis 122 by engaging with the rim 152 (see FIG. 20). The piezoelectric assembly 110 is disposed above a refill cavity 240, and is configured to receive the distal end 204 of the wick 188 when the refill 52 is inserted into the chassis 122.

Referring now to FIG. 14, another cross-sectional view of the dispenser 50 is shown. The wire tube 222 and the wires 226 are shown in cross-section, and a plurality of LEDs 218 are shown being disposed above the PCB 216. Further, additional refill guide ribs 230 are shown, which partially define a profile of the head 176 of the refill 52, and are configured to snugly receive the refill 52 within the chassis 122. The spring 232 comprises a top end 242 and a bottom end 244. The piezoelectric assembly 110 is also shown in FIG. 14, and is disposed below the bottom end 244 of the spring 232. The bottom end of the spring 232 may be fixedly attached with the piezoelectric assembly 110, or may be separate but disposed in physical contact with one another.

The bottom end 244 of the spring 232 is formed to receive the piezoelectric assembly 110, which is in turn formed to receive the upper wick 192 when the refill 52 is engaged therewith. The top end 242 of the spring 232 is wrapped around and secured to the cylindrical wall 164 of the crown 124. The chassis 122 includes an outer ledge 250 that is engaged with an inner ledge 252 of the crown 124. The chassis 122 and the crown 124 are snap fit together, however, the chassis 122 and the crown 124 may be coupled together in another fashion, such as via adhesive, fasteners, an interference fit, or a friction fit. Fastener walls 254 defining two of the fastener channels 214 are also shown clearly in FIG. 14, the fastener walls 254 extending between the bottom wall 92 of the base 56 and the platform 118.

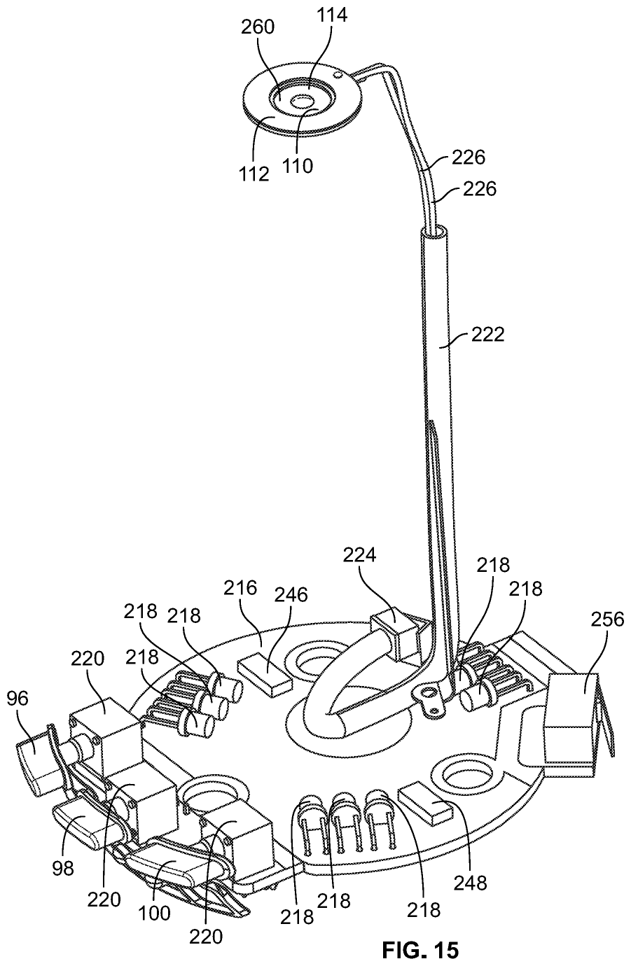

Referring to FIG. 15, the printed circuit board (PCB) 216, the wire tube 222 and the piezoelectric assembly 110 are shown in an isometric view. A controller 246 and a timer 248 are shown in schematic view along the PCB 216. The controller 246 may be a microcontroller, and may be disposed within or along the PCB 216. The controller 246 may also be a separate component that is electrically coupled with the PCB 216. The timer 248 may further be disposed within or along the PCB 216, or may be a separate component. The timer 248 is used to time when the dispenser 50 is activated, while the controller 246 is operable to receive instructions from the buttons 96, 98, 100 to activate the piezoelectric assembly 110, the timer 248, and/or the LEDs 218. As noted above, the PCB 216 is disposed between the base 56 and the platform 118, which are both removed from the view of FIG. 15 for clarity.

The LEDs 218, the buttons 96, 98, 100, the switches 220, and the wires 226 are shown clearly in the view of FIG. 15. A limit switch 256 is further shown, which provides a signal to the controller 246 that the shroud 54 has been secured to the base 56, and does not allow the dispenser 50 to be activated until the shroud 54 has been secured to the base 56. In some embodiments, there is no limit switch 256. The terminal 224 is further shown disposed along the PCB 216. Other electrical components, such as resistors, transistors, capacitors, processors, controllers, and relays may further be disposed along or within the PCB 216. In some embodiments, a plurality of batteries (not shown) may be enclosed within a casing (not shown) and may be electrically connected to the PCB 216 for providing electrical power to the PCB 216 and other electrical components of the dispenser 50.

Referring to FIG. 16, a top view of the PCB 216 is shown with the wire tube 222 and the piezoelectric assembly 110 having been removed for clarity. The switches 220 are shown in greater detail, and are shown positioned adjacent each of the first button 96, second button 98, and third button 100. Further, three sets of three LEDs 218 each are shown in detail in a triangular configuration along the PCB 216. The different sets of LEDs 218 may emit different colored lights, or may emit the same color. Further, the LEDs 218 may have the same intensity or a different intensity from one another. The port 104 is further shown along the PCB 216 at an opposite end of the second button 98. The PCB 216 has a generally circular profile; however, the PCB 216 may have any profile that allows it to be securely retained within the profile of the shroud 54.

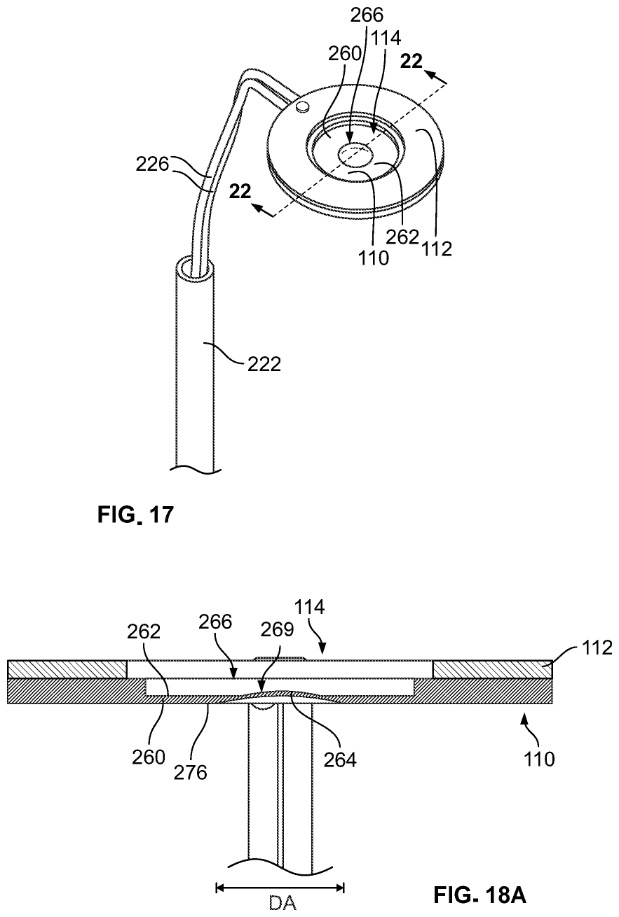

Referring now to FIG. 17, the piezoelectric assembly 110 is shown in greater detail, along with the wires 226 and an upper end of the wire tube 222. The piezoelectric assembly 110 includes a piezoelectric plate 260 and the piezoelectric element 112 that circumscribes the periphery of an upper surface 262 of the piezoelectric plate 260. The piezoelectric plate may comprise stainless steel, which may be SUS 316L steel. When the piezoelectric element 112 is energized, the piezoelectric plate 260 is caused to expand and contract, thereby releasing a volatile into the surrounding environment. The piezoelectric plate 260 further includes a central dome 264 that is generally concave and circular in shape. The piezoelectric assembly 110 includes an apertured section 266, at least a portion of which extends along the central dome 264. In an illustrative embodiment, the apertured section 266 of the piezoelectric plate 260 includes a plurality of orifices 268 having a diameter or at least one width dimension of between about 3 microns and about 9 microns, or between about 5 and 7 microns, or about 6.5 microns. In other illustrative embodiments, the piezoelectric plate 260 includes a plurality of orifices having a diameter of between about 3 microns and about 5 microns.

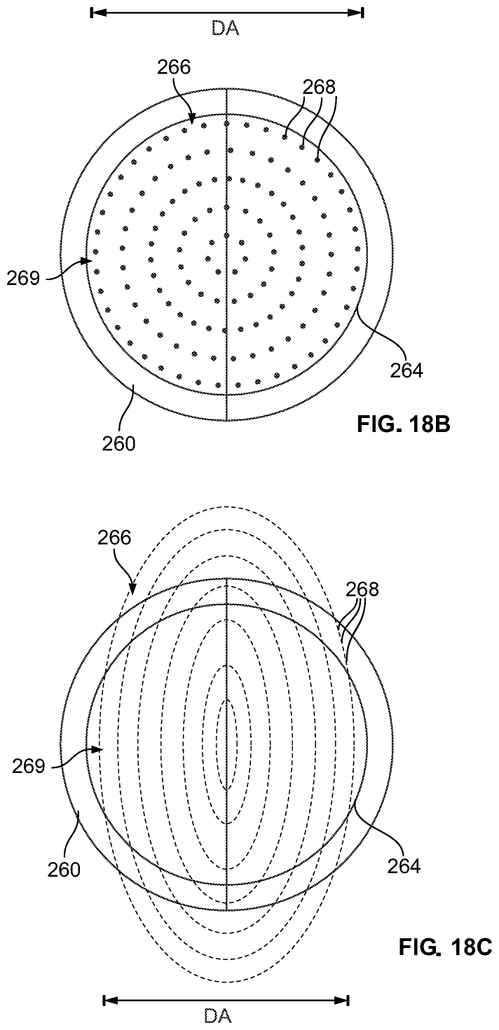

Referring to FIGS. 17 and 18A, the piezoelectric assembly 110, when in use, is positioned adjacent the wick 188. In illustrative embodiments, the piezoelectric element 112 may be formed as a ring and may be made of ceramic. In alternative illustrative embodiments, the piezoelectric assembly 110 may be formed in any suitable shape and/or may be made of any suitable material having piezoelectric properties and which causes the material to change dimensionally in response to an applied electric field. Illustrative examples of suitable materials include, but are not limited to, lead zirconate titanate (PZT) or lead metaniobate (PN). While a particular piezoelectric element is described, any actuator may be utilized, for example, a piezoelectric vibrating mesh actuator, a piezoelectric standing wave actuator, a piezoelectric vibrating needle, or any other suitable piezoelectric actuator.

Referring to FIGS. 18B and 18C, the plurality of orifices 268 along the piezoelectric plate 260 may define an array 269 that is circular in nature. The array 269 of orifices may be defined along the entire dome 264, as shown in FIG. 266. The array 269 may alternatively be disposed along only a portion of the dome 264, or may extend beyond the dome 264, as depicted in FIG. 18C. The array 269 may have alternative configurations, and may be in the shape of a triangle, a square, an oval, or a polygon. Further, the orifices 268 may be in the shape of a circle (FIG. 18B), a rectangle or square (FIG. 18C), or another shape. It should be noted that the orifices 268 depicted in the schematic views of FIGS. 18B and 18C do not represent the actual size or actual number of the orifices 268, rather, the orifices 268 may define dimensions as noted herein.

In some embodiments, the array 269 has a diameter or at least one width dimension of between about 0.5 mm and about 10.0 mm, or between about 1.0 mm and about 9.0 mm, or between about 2.0 mm and about 8.0 mm, or between about 3.0 mm and about 7.0 mm. Referring again to FIG. 12, a wick diameter DW and an array diameter DA (see FIG. 18) may define a ratio of DW/DA of between about 1.0 and about 3.0. In some embodiments, the ratio is about 1.1. In some embodiments, the wick diameter DW is between about 3.0 mm and about 5.5 mm, or between about 3.5 mm and about 4.0 mm, or about 3.8 mm. In some embodiments, the array diameter DA is between about 3.0 mm and about 6.0 mm, or between about 4.0 mm and about 5.0 mm, or about 4.5 mm. The ratio of wick diameters may affect the accuracy and consistency of the plume of the volatile that is emitted by the dispenser 50. For example, having a ratio DW/DA of about 1.1 has been found to create a repeatable and accurate dispersal of volatile from the dispenser 50. In some embodiments the array 269 may comprise between about 100 and about 400 orifices, or between about 150 and about 350 orifices, or about 316 orifices, or about 200 orifices.

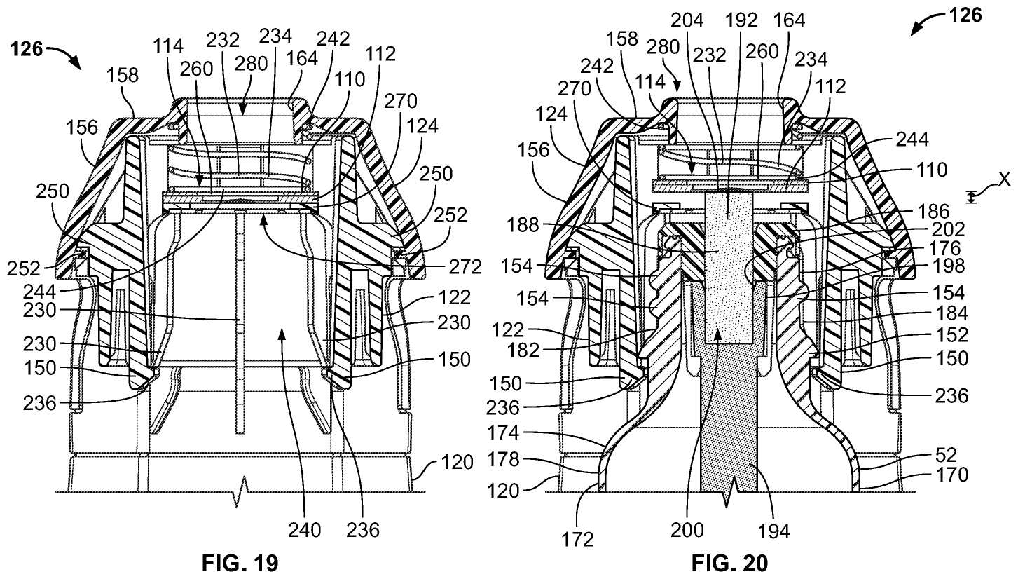

Referring now to FIGS. 19 and 20, detail views of the upper end of the stand assembly 116 are shown without and with a refill having been inserted therein, respectively. Referring to FIG. 19, the piezoelectric assembly 110 is shown in greater detail. In some embodiments, a soft material, such as loading foam, may be provided along an underside of the piezoelectric assembly 110. Additional materials may be provided along an underside of the piezoelectric assembly 110 to aid in accuracy or consistency of the plume of volatile generated when the dispenser 50 is activated. The additional materials may also be provided to enhance or reduce a dampening effect that could be caused by the spring 232, the wick 188, and/or the piezoelectric plate 260.

The top end 242 of the spring 232 is shown wrapped around the cylindrical wall 164, while the bottom end 244 of the spring 232 is shown in contact with and applying a force against the piezoelectric assembly 110. The chassis 122 further includes a piezo platform 270, which is unitary with the chassis 122 and defines a surface to which the piezoelectric assembly 110 is secured. The piezo platform 270 retains the piezoelectric assembly 110 in place, and prevents the piezoelectric assembly 110 from being displaced farther than the piezo platform 270. The piezo platform 270 is generally circular and includes an aperture 272 in a center thereof, which allows the distal end 204 of the wick 188 to engage in physical contact with the piezoelectric assembly 110 when inserted into the chassis 122. The spring 232 is positioned to provide an opposing force against the refill 52 when it has been inserted into the chassis 122. As noted below, when the wick 188 is engaged with the piezoelectric assembly 110, the spring 232 is compressed and the piezoelectric assembly 110 is displaced a distance X above the piezo platform 270. This displacement is discussed in greater detail hereinafter below.

Referring to FIG. 20, the refill 52 is shown having been inserted into the manifold 126. Because the refill 52 has been inserted into the refill cavity 240 of the chassis 122, the spring 232 has been compressed, thus, the spring 232 is shown in a compressed configuration. Still referring to FIG. 20, the rim 152 of the refill 52 is also shown engaged with the catches 236 of the tabs 150, and the distal end 204 of the wick 188 is shown in physical contact with a lower surface 276 (see FIG. 18) of the piezoelectric assembly 110. The compression of the spring 232 shown in FIG. 20 is a result of the size and type of spring that is used in the dispenser 50, which may be chosen based upon a variety of factors, as discussed below. Other than the spring 232 having been compressed, the piezoelectric assembly 110 having been translated upward due to the force applied by the wick 188, and the catches 236 of the tabs 150 having been engaged with the rim 152 of the refill 52, no other elements of the manifold 126 are moved or manipulated when the refill 52 is inserted into the manifold 126. FIG. 20 shows the refill 52 in an operational state, where the dispenser 50 could be activated to disperse the volatile by providing an electrical pulse to the piezoelectric assembly 110.

In illustrative embodiments, and as noted above, an absorbent material (not shown) may be included between the wick 188 and the piezoelectric assembly 110. The absorbent material may be a felt pad and/or cotton wool. In other illustrative embodiments, the absorbent material may be formed of a velour pad, cotton cloth, chenille yarn, chenille fabric, polyester cloth, paper towel, synthetic cloth, synthetic nonwoven material, a cotton ball or swab, combinations thereof, or other suitable absorbent material(s). The absorbent material may be a component of the nebulizer assembly or may be attached or otherwise in communication with the wick 188 of the refill 52.

During operation, the piezoelectric assembly 110 is actuated, either continuously or intermittently, to dispense volatile material. More particularly, an oscillating electric field is applied to the piezoelectric element 112, which causes expansion and contraction of the piezoelectric plate 260 in a radial direction. The expansion and contraction causes the piezoelectric plate 260 to vibrate in an axial direction (along a longitudinal axis of the dispenser 50), forcing volatile material retained within the orifices of the piezoelectric plate 260 away from the piezoelectric assembly 110, through a channel 280 defined by the cylindrical wall 164, and through the chimney 78 of the shroud 54.

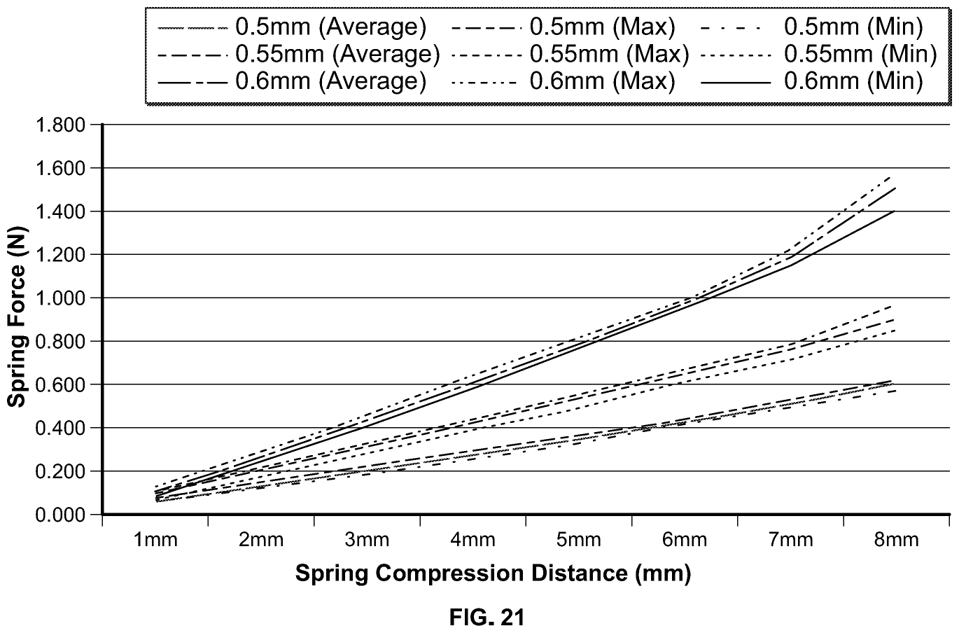

Referring to the graph of FIG. 21, three separate groups of springs were tested as the spring 232 in the dispenser 50 described above, the results of which are illustrated in the figure. The three groups of springs comprise spring wires 234 having a diameter of 0.50 mm, a spring having a diameter of 0.55 mm, and a spring having a diameter of 0.60 mm. The graph illustrates a spring force measured in Newtons (N) against a spring compression distance measured in millimeters (mm). The spring compression distance is the distance X noted above, i.e., a distance between the piezo platform 270 and the piezoelectric assembly 110 after the wick 188 causes the piezoelectric assembly 110 to be translated upward. The graph further illustrates a minimum, an average, and a maximum for each of the three springs. Testing involved iteratively compressing springs from 1.0 mm to 8.0 mm while recording the resulting force output (N). While the three different spring diameters, i.e., 0.50 mm, 0.55 mm, and 0.60 mm, achieved different results, the results suggest that at a compression of 7.0 mm, each of the three springs saw a non-linear increase in measured spring force.

In some embodiments, the spring 232 may have an uncompressed height of between about 7 mm and about 20 mm, or between about 10 mm and about 17 mm, or about 13 mm. Further, in some embodiments, the spring 232 may have between about 2 turns and about 10 turns, or between about 3 turns and about 7 turns, or about 4.5 turns. In a preferred embodiment, the spring 232 has a spring wire diameter of about 0.6 mm, an uncompressed height of about 13 mm, and comprises about 4.5 turns.

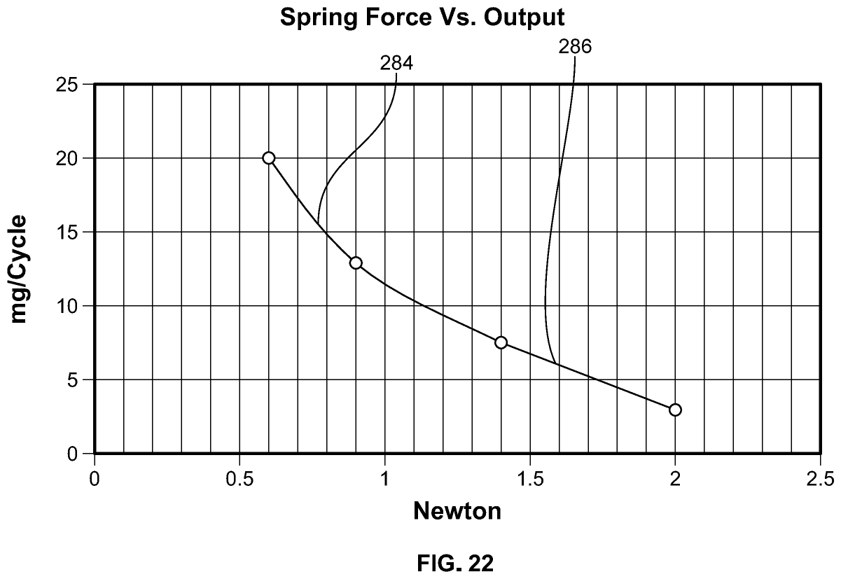

Referring now to the graph of FIG. 22, the spring force measured in Newtons (N) against a fragrance output measured in milligrams per cycle (mg/cycle) is shown. The graph illustrates results from the testing displayed in FIG. 21. The results of the tests suggest that the dispenser 50 emits a greater volume of fragrance when the springs induce lower compressive forces upon the piezoelectric plate 260. Fragrance emission patterns can be simplified into a first linear region 284 and a second linear region 286. The first region 284 occurs between 0.6 and 0.9 N and has an approximate slope of −25 mg/cycle/N. The second region 286 occurs between 0.9 and 2.0 N and has an approximate slope of −8.33 mg/cycle/N. The results suggest that the fragrance device emits a larger volume of fragrance when subjected to lower spring forces. As a result, depending on the desired range of outputs, a particular spring may be identified for use with the dispenser 50. To that end, the 0.50 mm or 0.55 mm spring would likely be utilized if one desired to achieve a fragrance output within the first linear region 284 and the 0.60 mm spring would likely be utilized if one desired to achieve a fragrance output within the second linear region 286.

Based on the testing, it was determined that having an upper wick 192 with a more pliable material causes less displacement of the spring 232 that applies a force against the piezoelectric assembly 110, which results in a more predictable dispersal of volatile. The variability of the dispensed plume is more predictable when operating within the linear region, while the variability of the release rate increases significantly when the force displacement of the spring 232 is non-linear. It has also been found that the amount of force that is applied by the spring 232 against the piezoelectric assembly 110 has a direct correlation with the release rate of the volatile. The lower the force, the more the piezoelectric assembly 110 can bounce within the channel 280.

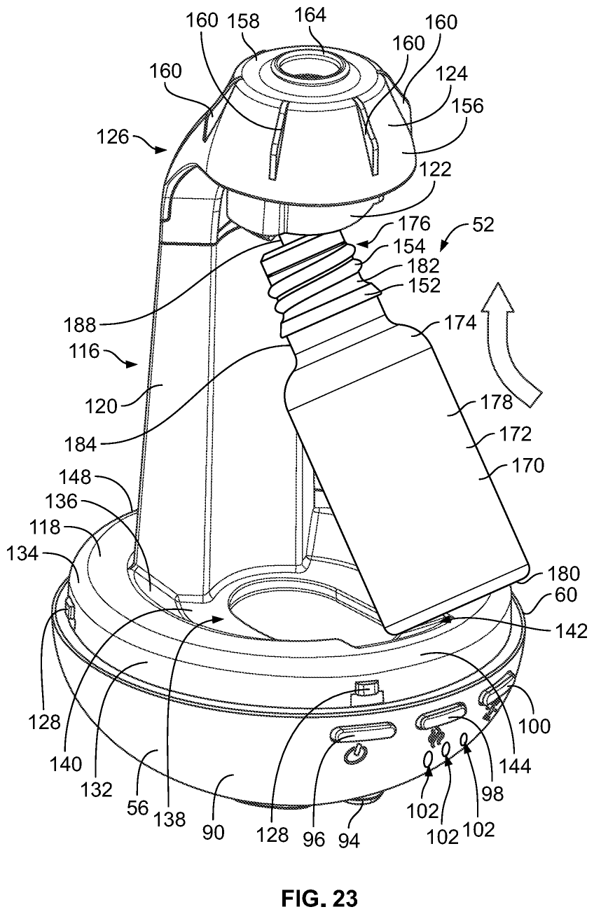

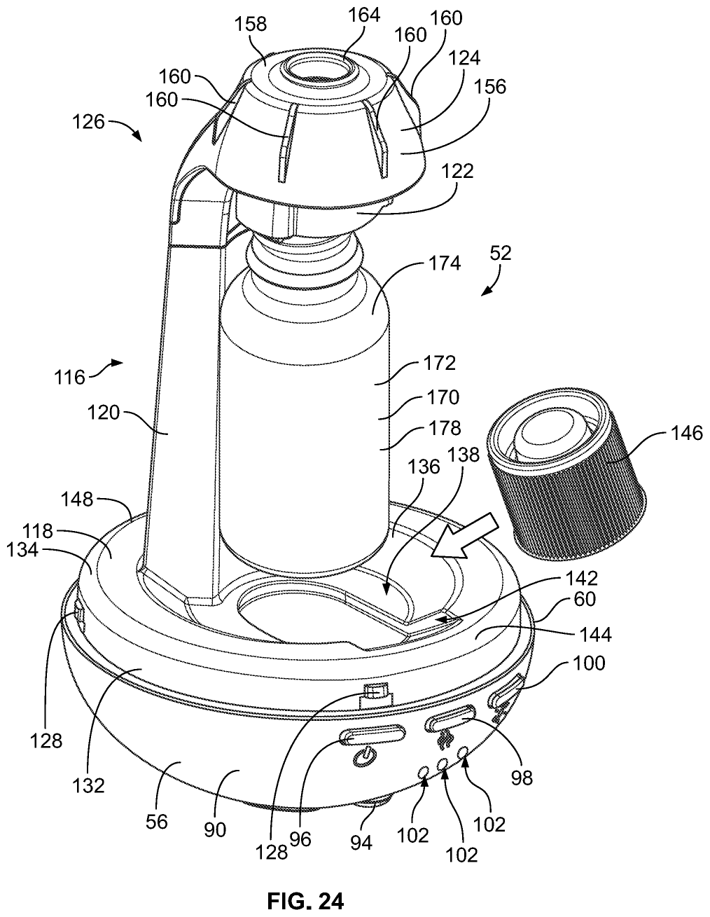

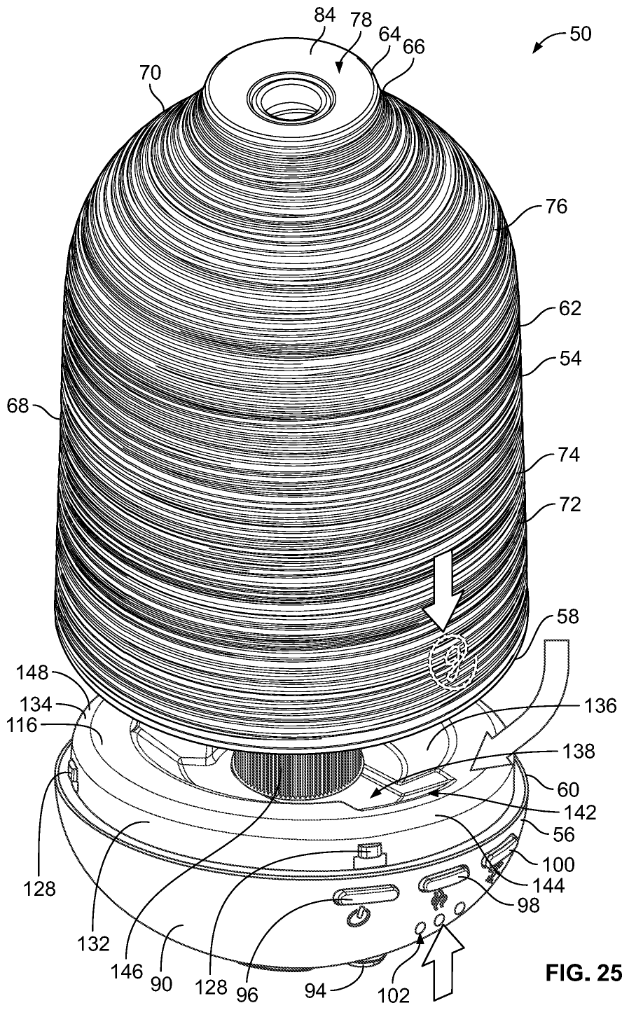

Referring to FIGS. 23-25, a method of inserting the refill 52 into the stand assembly 116 and assembling the dispenser 50 are depicted. Referring to FIG. 23, the refill 52 is positioned with the head 176 thereof being inserted into the manifold 126. The lower wall 180 of the refill 52 is slid through the slot 142 of the platform 118 and the refill 52 is moved upward so that the head 176 is inserted into the manifold 126. Referring to FIG. 24, once the rim 152 of the refill 52 has been engaged with the catches 236 of the tabs 150, the refill 52 can be characterized as being secured within the manifold 126. The cap 146 of the refill 52 may be inserted into the well 138 to securely retain the cap 146 while the refill 52 is in use. Referring to FIG. 25, the shroud 54 is aligned with the base 56 and is rotated until the protrusions 128 along the base 56 engage with features along the underside 162 of the shroud 54. The shroud 54 may alternatively be engaged with the base 56 in another way. Once the shroud 54 has been coupled with the base 56, one end of a power cord (not shown) is plugged into the port 104 and the other end is plugged into a power source such as a power adapter, a laptop, or another low voltage outlet.

Still referring to FIG. 25, the first button 96 is a power button, the second button 98 is a fragrance strength button, and the third button 100 is a light button. The first button 96 may be pressed once to turn the dispenser on. The dispenser 50 will begin to dispense fragrance once the first button 96 has been depressed. The second button 98 and the third button 100 may be pressed to personalize the preference of the fragrance strength and/or the light scheme. During operation of the dispenser 50, it is desirable to have the wick 188 in contact with the piezoelectric plate 260. If the wick 188 is not in contact with the piezoelectric plate 260, especially in a water-based fragranced volatile material, the dispenser 50 may not function properly and/or may not emit volatile material at all.

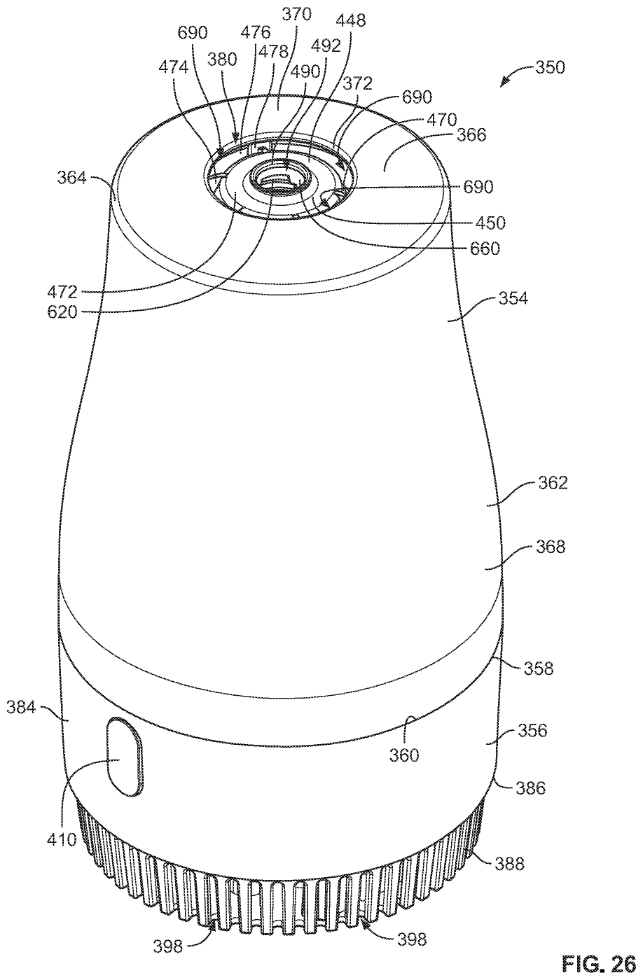

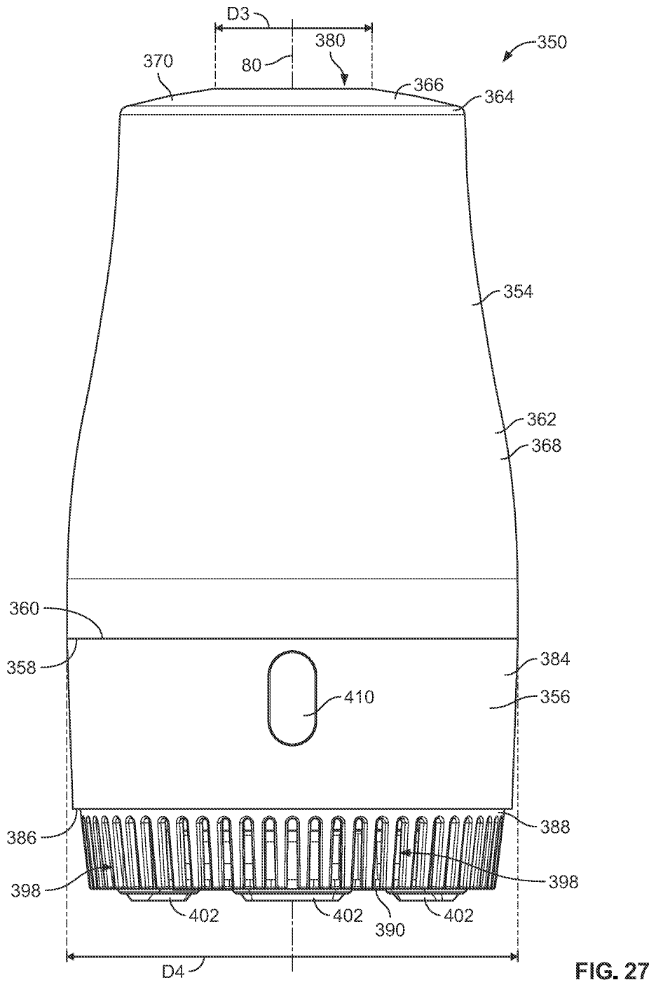

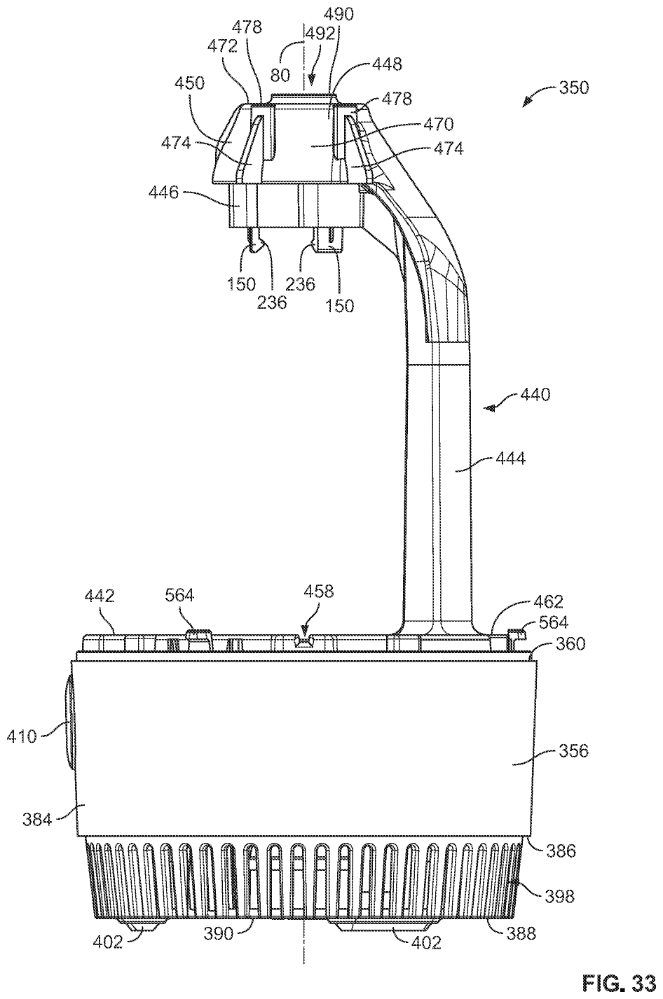

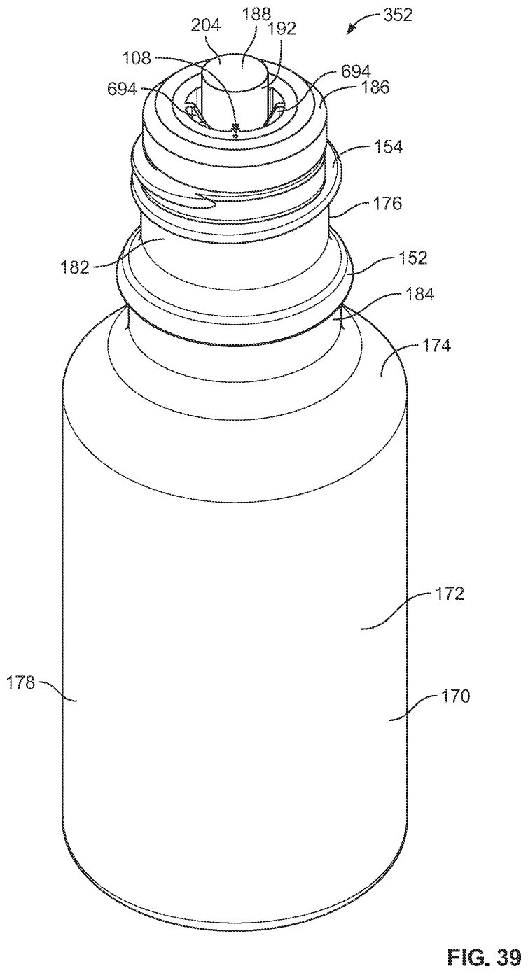

Referring to FIGS. 26-41, like reference numbers are used with regard to an alternative embodiment of a volatile material dispenser 350. The dispenser 350 may be adapted to accommodate a refill 352 (see FIG. 39) and dispense a volatile material in the form of water and/or fragranced oil from the refill 352, similar to the dispenser 50. In some embodiments, the dispenser 350 may dispense a pest control volatile material from the refill 352. Referring specifically to FIGS. 26 and 27, the dispenser 350 includes a shroud or cover 354 and a base 356. The shroud 354 defines a lower edge 358 that interfaces with an upper edge 360 of the base 356. A sidewall 362 of the shroud 354 extends upward from the lower edge 358 toward a rounded corner 364 at an upper end 366 of the shroud 354. The sidewall 362 of the shroud 354 defines a spline-shaped lower portion 368 and a frustoconical upper portion 370 that terminates at a lip 372. As illustrated in FIG. 27, the rounded corner 364 defines the boundary between the spline-shaped lower portion 368 and the frustoconical upper portion 370. The shroud 354 further includes a chimney 380 that is centered about the central longitudinal axis 80 (see FIG. 27). As noted herein, the chimney 380 is defined as the portion interior to the lip 372. In some embodiments, similar to the dispenser 50, the shroud 354 and the base 356 may comprise polypropylene (PP), however, the shroud 354 and the base 356 may comprise a wide variety of polymeric materials. In some embodiments, the shroud 354 may be transparent or translucent.

Referring still to FIGS. 26 and 27, the frustoconical upper portion 370 of the shroud 354 extends upward and away from the rounded corner 364. In other words, the frustoconical upper portion 370 extends upward and toward the longitudinal axis 80. Referring specifically to FIG. 27, while the chimney 380 may define a variety of diameters, the chimney 380 is illustrated having a diameter D3 that is about 33% of a widest diameter D4 of the shroud 354 taken at the lower edge 358 thereof In some embodiments, the diameter D3 may be between about 5% and about 70% of the diameter D4, or between about 10% and about 50% of the diameter D4, or between about 20% and about 40% of the diameter D4, or between about 30% and about 40% of the diameter D4, or between about 30% and about 35% of the diameter D4. In some embodiments, the diameter D3 is about 32 mm and the diameter D4 is about 98 mm.

Referring still to FIGS. 26 and 27, the base 356 also defines a sidewall 384 that extends from the upper edge 360 of the base 356 downward, toward a step 386 thereof. The sidewall 384 of the base 356 is tapered, and extends from the upper edge 360 downward and inward, toward the longitudinal axis 80. As illustrated in FIGS. 26 and 27, the base 356 also comprises a lower portion 388 that extends from the step 386 and comprises a bottom wall 390. The lower portion 388 of the base 356 comprises a plurality of vents 398 therein. As will be discussed in further detail herein, the plurality of vents 398 allow air to flow through the dispenser 350 and help to disperse the volatile material within the refill 352. As illustrated in FIG. 27, a plurality of feet 402 extend downward from the bottom wall 390, and are provided to allow the dispenser 350 to rest upon a flat surface (not shown). Further, a button 410 extends outwardly from the base 356, away from the longitudinal axis 80. The button 410 may be used for a variety of purposes, and may have a variety of different functions, as discussed hereinafter below.

Referring to FIG. 27, the button 410 may be provided along the base 356 of the dispenser 350. The button 410 may be configured to turn the dispenser 350 on and adjust the settings of the device. In some embodiments, the button 410 or portions surrounding the button 410 may illuminate. As will be discussed in more detail below, the button 410 can allow the user to turn on and off certain features of the dispenser 350. In other words, the button 410 can be used to adjust the settings of the dispenser 350. As illustrated in FIG. 27, the button 410 comprises a generally racetrack shape. However, it is contemplated that the button 410 can be any shape or size. Further, in some embodiments, the dispenser 350 may comprise more than one button 410, similar to the dispenser 50.

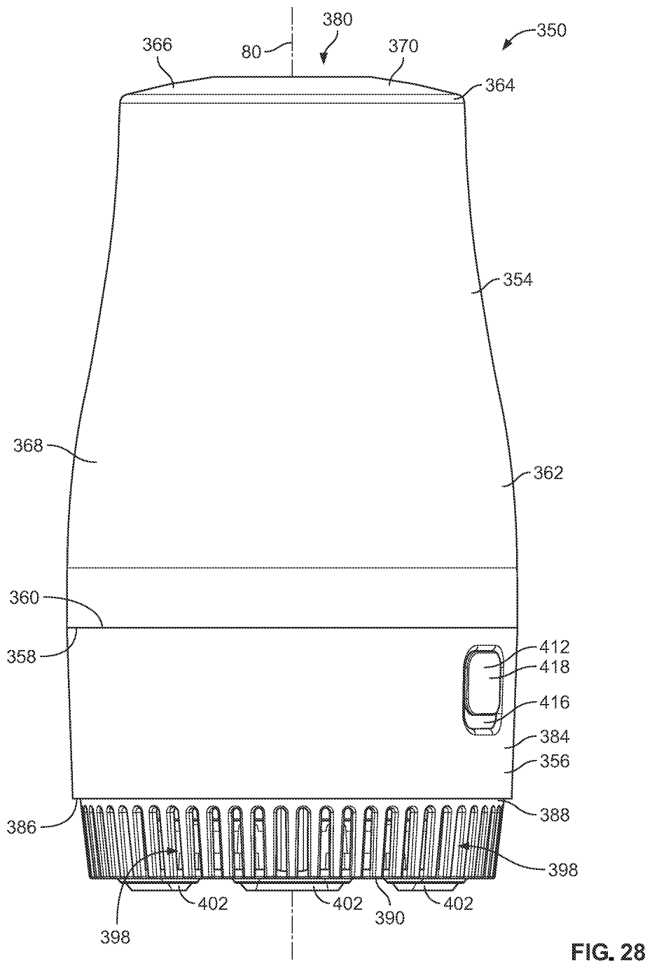

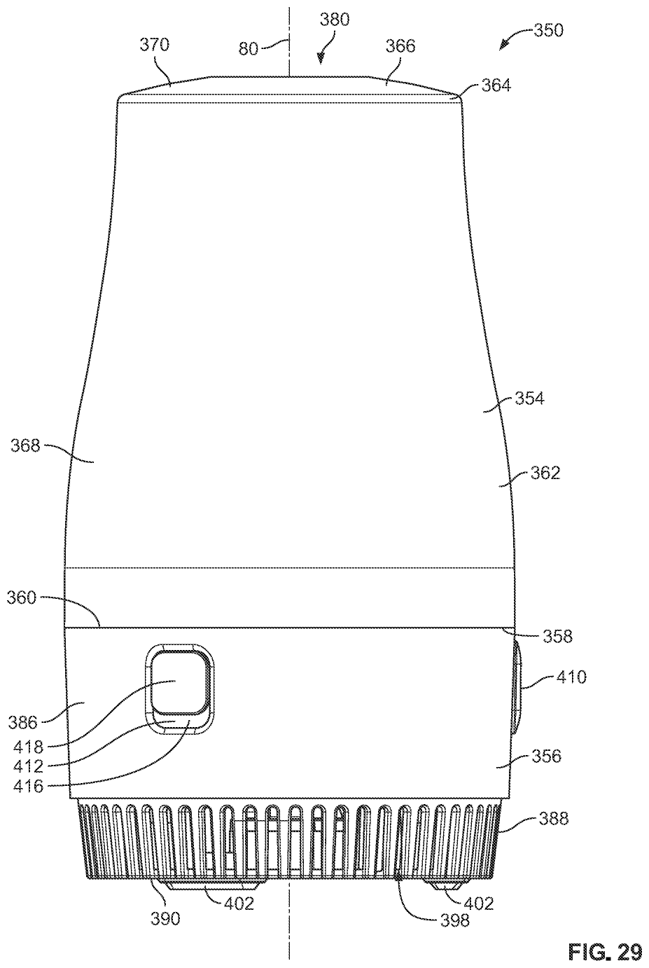

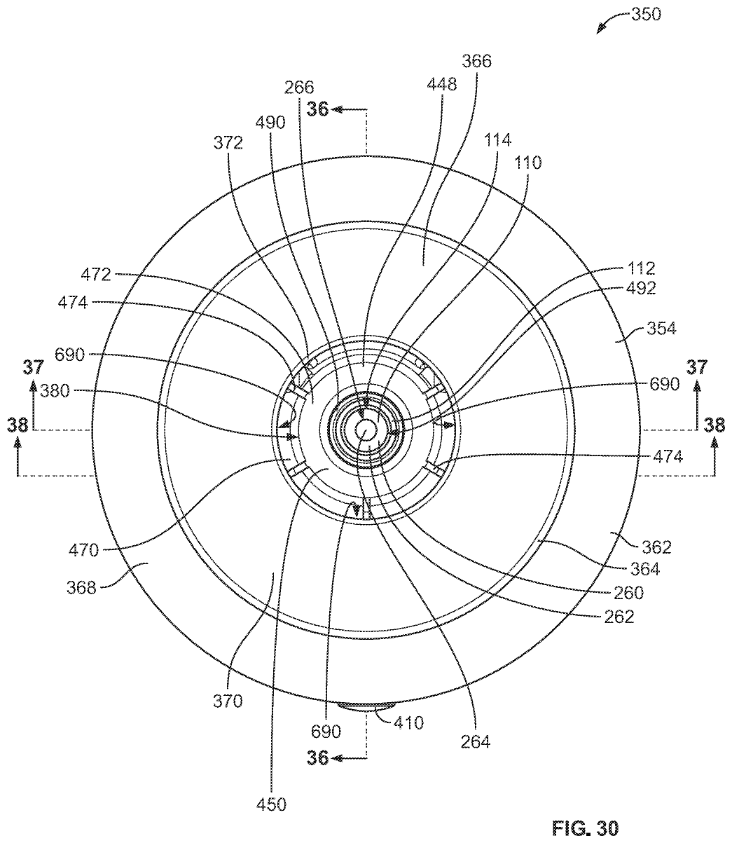

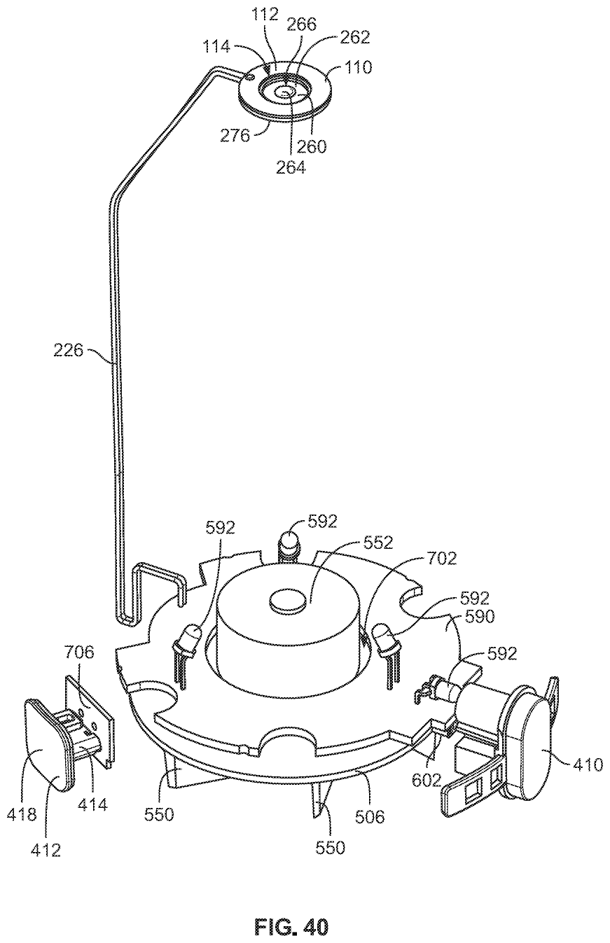

Referring now to FIGS. 28 and 29, a rear view and a right side view of the dispenser 350 is shown, respectively. As illustrated in FIGS. 28 and 29, the dispenser 350 further comprises a port assembly 412 that includes a low voltage receptacle or port 414 (see FIG. 40) for receiving a low-voltage electrical connector of a low voltage wire, such as a USB cord (not shown). The port assembly 412 also includes a recessed wall 416 and a port cover 418 that covers the port 414. The port cover 418 is configured to be removably attached to the port 414 in order to allow access therein. As noted herein, the port cover 418 is configured to protect the port 414 when the port 414 is not in use. In some embodiments, electrical prongs, a cord, or another suitable electrical connector may be electrically coupled with the dispenser 350 so as to allow electrical power to be provided to the dispenser 350. In some embodiments, the port 414 may be a USB-c port and/or be used to charge the dispenser 350. Besides for the port assembly 412, the base is symmetric about a plane 36-36 (see FIG. 30) that bisects the button 410. As further noted herein, additional features may be provided within or along the shroud 354 and/or the base 356. Furthermore, additional bodies defining one or more sidewalls (not shown) may be provided between, above, or below the base 356 and the shroud 354.

Referring now to FIG. 30, a top view of the dispenser 350 is depicted. The chimney 380 is shown in greater detail, which is centered along the longitudinal axis 80 (see FIG. 27). The piezoelectric assembly 110 is shown within the chimney 380, which is also centered along the longitudinal axis 80. The annular piezoelectric element 112 is also visible through the chimney 380. As discussed above with respect to the dispenser 50, the piezoelectric element 112 includes the central aperture 114 through which liquid volatile within the refill 352 is dispersed when the dispenser 350 is activated. When the dispenser 350 is activated, a stream/plume of the volatile is dispensed through the chimney 380 of the shroud 354 and out of the dispenser 350, as discussed in greater detail hereinafter below. In contrast to the chimney 78, the chimney 380 is larger and allows more airflow therethrough. As will be further discussed below, volatile material from the refill 352 and air flowing through the dispenser 350 (via the plurality of vents 398) can move through the chimney 380.

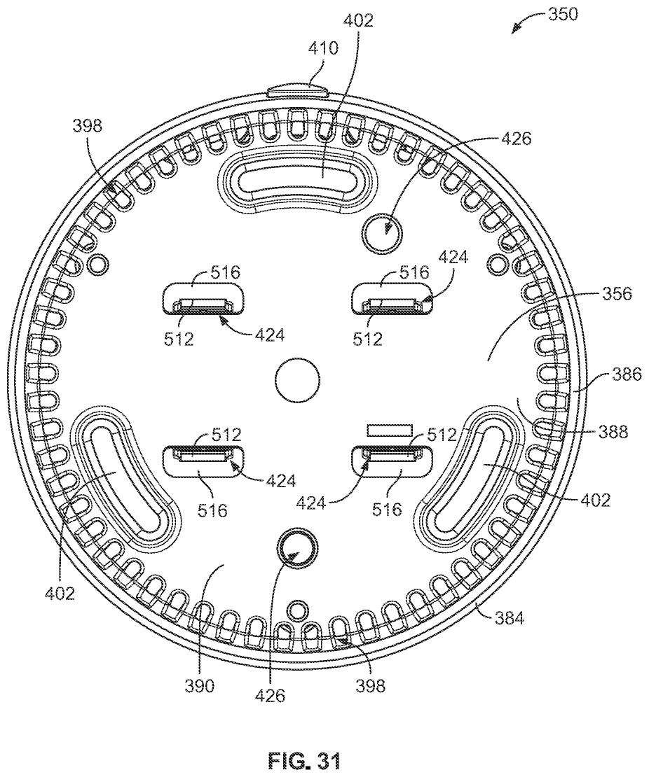

Referring now to FIG. 31, the bottom wall 390 of the lower portion 388 of the base 356 is shown in greater detail. The plurality of feet 402 extend from the bottom wall 390. In preferred embodiments, the base 356 comprises three feet 402. However, in some embodiments, 1, 2, 3, 4, 5, 6, 7, 8, 9, or more feet may extend form the bottom wall 390. As illustrated in FIG. 31, the plurality of feet 402 comprise a generally oval or race track shape that is canted or angled. In other words, the plurality of feet 402 are rotationally symmetric about the longitudinal axis 80. Alternative configurations or shapes of the feet 402 are contemplated, while in some embodiments additional structure may be included in addition to the feet 402. In some embodiments, there are no feet 402 or other types of stability mechanisms that retain the dispenser 350 in an upright configuration.

Referring still to FIG. 31, the bottom wall 390 also includes a plurality of slots 424 that extend through the bottom wall 390 and into the dispenser 350. Further, the bottom wall 390 includes a plurality of cylindrical slots 426 extending into the dispenser 350. The plurality of slots 424 help secure internal components to the bottom wall 390 and the plurality of cylindrical slots 426 help support the base 356 and portions therein. As illustrated in FIG. 31, the plurality of vents 398 also extend onto a periphery of the bottom wall 390.

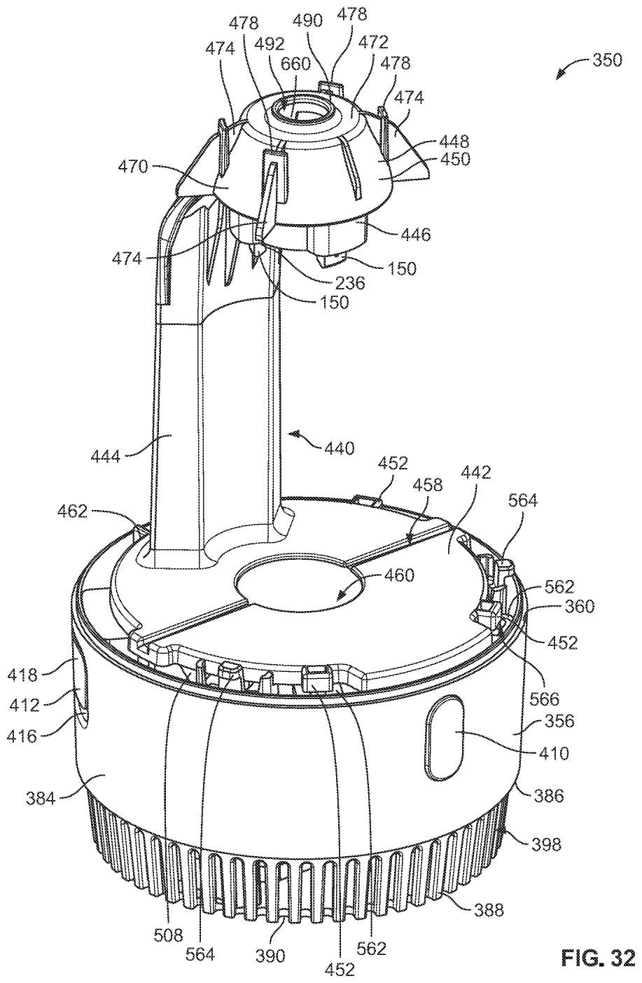

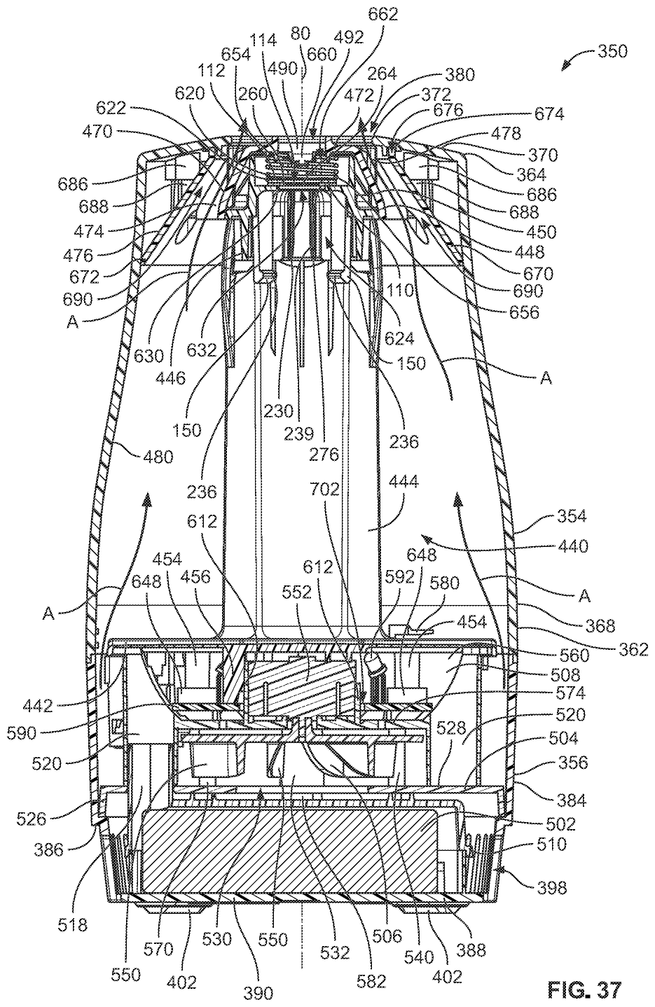

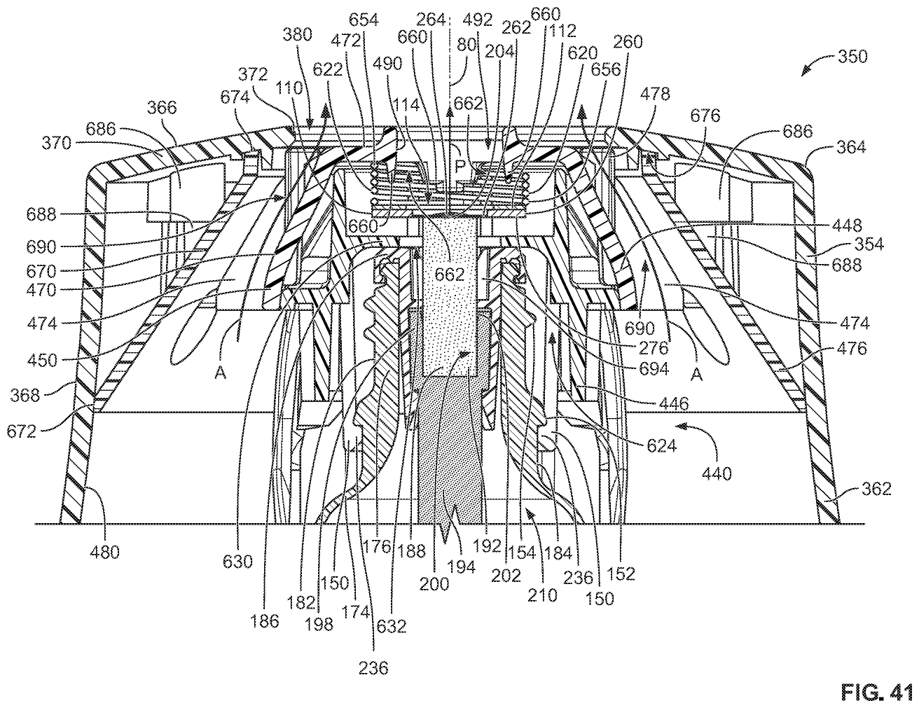

Referring now to FIGS. 32-34, a stand assembly 440 of the dispenser 350 is shown, with the shroud 354 having been removed for clarity. Similar to the dispenser 50, the stand assembly 440 includes a platform 442, a stand 444, a refill chassis 446, and a crown 448. The refill chassis 446 and the crown 448 define a manifold 450 that retains the refill 352 and the piezoelectric assembly 110 (see FIG. 41). As illustrated in FIGS. 32-34, the platform 442 extends upward from the base 356 and is inset from the sidewall 384 thereof. As will be discussed in further detail herein, the platform 442 comprises a plurality of hooks 452 that secure with base 356. Once assembled, the platform 442 is securely retained on the base 356 by the plurality of hooks 452 and a plurality of platform posts 454 that extend downwardly from the platform 442 (see FIG. 37). In some embodiments, the platform 442 may further comprise a plurality of platform ribs 456 that secure the platform 442 to the base 356 and provide additional support thereto (see FIG. 37). Further, in some embodiments, the platform 442 may be permanently or removably coupled with the base 356 via a plurality of fasteners or fastening mechanisms, such as fasteners, rivets, nails, bolts, or cables.

Referring still to FIGS. 32-34, the platform 442 comprises a generally circular shape with a groove 458 running through the center. The platform 442 also comprises a well 460 at the center of the platform 442. The well 460 comprises a circular shape and may assist in the insertion of the refill 352 into an operable configuration. The well 460 may also include additional features that aid in retaining the refill 352 or another component. For example, in some embodiments, the well 460 of the platform 442 may be sized and shaped to retain the cap 146 (see FIG. 10) of the refill 352 after the cap 146 has been removed from the refill 352.

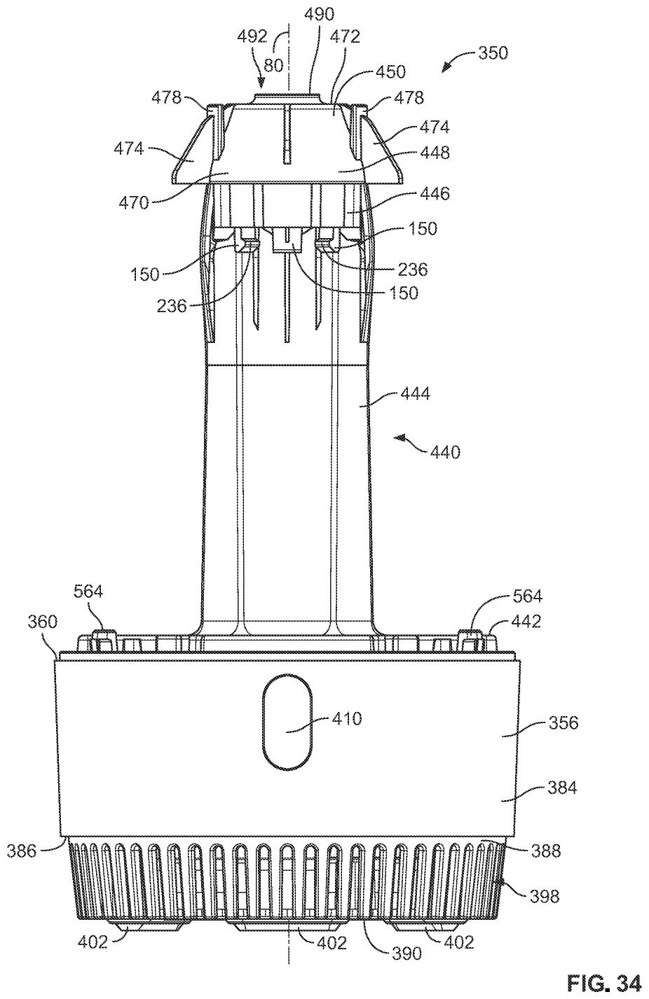

Referring still to FIGS. 32-34, the stand assembly 440 includes the stand 444, which extends upward from the platform 442 along a rear portion 462 thereof. The stand assembly 440 comprises a unitary piece that extends upward from the platform 442 toward the manifold 450, which extends inwards, and aligns with the longitudinal axis 80. Elements of the stand assembly 440 may also comprise polypropylene or another type of polymeric material. As noted above, the manifold 450 includes the crown 448 and the refill chassis 446, which extends downward from the crown 448. Similar to the chassis 122 described above, the chassis 446 includes the tabs 150 that depend downward to retain the annular rim 152 (see FIG. 41) of the refill 352. However, as illustrated in FIG. 34, the chassis 446 includes three tabs 150 that extend downward from the chassis 446 as opposed to two tabs 150 of the chassis 122. The three tabs 150 are positioned rotationally offset from each other. In other words, the three tabs 150 are positioned 120° offset from each other with respect to the longitudinal axis 80. As discussed above, the tabs 150 may be formed to flex outward when the refill 352 is laterally inserted into the chassis 446 until the rim 152 of the refill 352 (see FIG. 41) engages with the tabs 150. In particular, the inwardly-depending catches 236 are configured to translate outward, away from the longitudinal axis 80 as the refill 352 is being inserted into the chassis 446, and snap back inward to secure the refill 352 in place within the chassis 446 by engaging with the rim 152 (see FIG. 41). In some embodiments, the chassis 446 may include 1, 2, 3, 4, 5, 6, 7, 8, 9, or more tabs 150 extending downward therefrom. Further, in some embodiments, the tabs 150 shown in FIGS. 32-34 may extend farther down from the chassis 446 than the tabs 150 shown in FIGS. 7-9 to accommodate the longer refill 352.

Similar to the refill 52, the refill 352 may be removed from the tabs 150 by a user grabbing the refill 352 and laterally pulling the refill down. In some embodiments, the forces required for insertion and removal of the refill 352 are low enough to simply allow for lateral insertion and removal. However, in some embodiments, the refill 352 may be removed by squeezing the tabs 150 to deflect the tabs 150 outward, thereby releasing the rim 152 from engagement therewith. In some embodiments, the refill 352 may be disengaged by rotating the refill 352 such that the threading 154 (see FIG. 39) allows the refill to be rotated and translated downward, away from the crown 448, for removal from the manifold 450. Alternative removal mechanisms may be implemented, such as one or more buttons (not shown) that could be depressed to cause the refill 352 to be released from the chassis 446.

With reference still to FIGS. 32-34, the crown 448 extends upward from the chassis 446 and defines a generally frusto-conical side surface 470 that terminates at a top wall 472. A plurality of ribs 474 are radially disposed along the side surface 470, which may be formed to aid in physical engagement with an air deflector 476 (see FIG. 41). The ribs 474 are spaced apart about the longitudinal axis 80 and extend farther outward from the side surface 470 of the crown 448 than the ribs 160 described above in connection with the dispenser 50. Each of the plurality of ribs 474 can comprise a support 478 that extends from the side surface 470 of the crown 448 and through the ribs 474. The supports 478 can contact an underside 480 of the shroud 354 (see FIG. 41). In some embodiments, the crown 448 may include more or fewer ribs 474 than shown.

With reference still to FIGS. 32-34, an annular wall 490 extends upward from the top wall 472 of the crown 448, the annular wall 490 having a center that is coaxial with the longitudinal axis 80. The annular wall 490 defines a channel or passageway 492 therethrough and is also coaxial with the chimney 380 defined by the shroud 354. As noted herein, the annular wall 490 is similar to the cylindrical wall 164 described above with respect to the dispenser 50. In some embodiments, the annular wall 490 may define a cylindrical surface. As further noted herein, the shroud 354, the platform 442, the stand 444 and the manifold 450 may be characterized as being symmetric about the plane 36-36 (see FIG. 30) that intersects the button 410 and extends through the longitudinal axis 80.

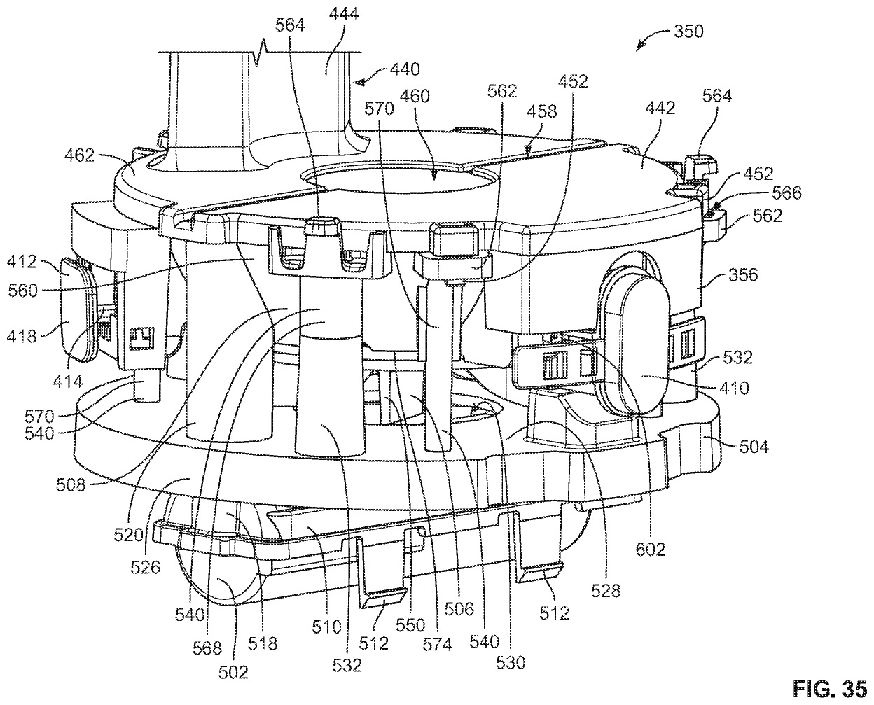

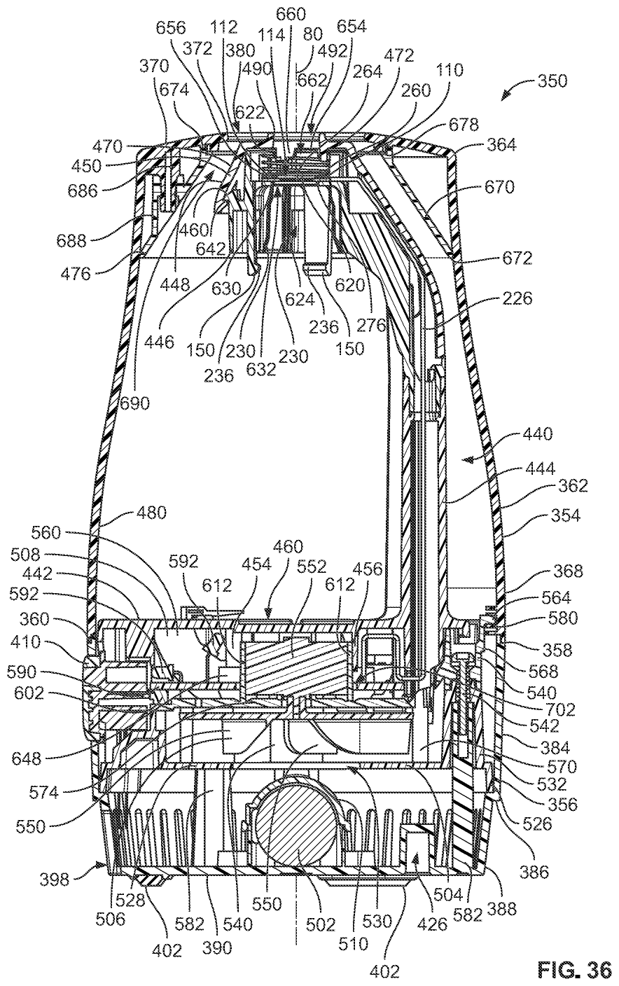

Referring now to FIG. 35, an interior of the base 356 is shown, with the sidewall 384 and the lower portion 388 of the base 356 removed for clarity. As illustrated in FIG. 35, the base 356 further includes a battery 502, an air isolator 504, a fan 506, and a sealed engine 508. The battery 502 is secured with a battery cover 510 and is positioned below the air isolator 504. The battery cover 510 includes a plurality of battery tabs 512 that extend downward from the battery cover 510, and the battery cover 510 is configured to protect the battery 502 from external elements, i.e., rain, dust, etc. During use, the plurality of battery tabs 512 are configured to extend through the bottom wall 390 of the base 356 via the plurality of slots 424 and secure with a bottom recessed portion 516 of the bottom wall 390 of the base 356 (see FIG. 31). In some embodiments, the battery cover 510 and portions of the lower portion 388 of the base 356 may securely encase the battery 502 (see FIGS. 36 and 37). In other words, the battery cover 510 and portions of the lower portion 388 of the base 356 extending from the bottom wall 390 may protect the battery 502 from external elements (see FIGS. 36 and 37).

Referring still to FIG. 35, the battery cover 510 also includes a battery cover post 518 that secures with a sealed engine support 520 of the sealed engine 508 (see FIG. 37). In preferred embodiments, the battery 502 is a rechargeable Li-ion battery and/or a NIMH battery. However, in some embodiments, the battery 502 may include Alkaline batteries. Therefore, it is contemplated that any type of battery may be used for the battery 502. As noted above, the battery 502 is a rechargeable battery 502 that may be charged via the port assembly 412. Therefore, in preferred embodiments, the battery 502 can be electrically coupled with the port assembly 412. In some embodiments, the battery 502 can allow the dispenser 350 to operate for 6-12 hours before needing to be charged or replaced. Further, in some embodiments, the battery 502 may be removably coupled with the base 356. Therefore, in some embodiments, the battery 502 can be removed from the base 356 and charged separately. Thus, the original battery with no power left can be replaced with a fully charged battery while the original battery is being charged.

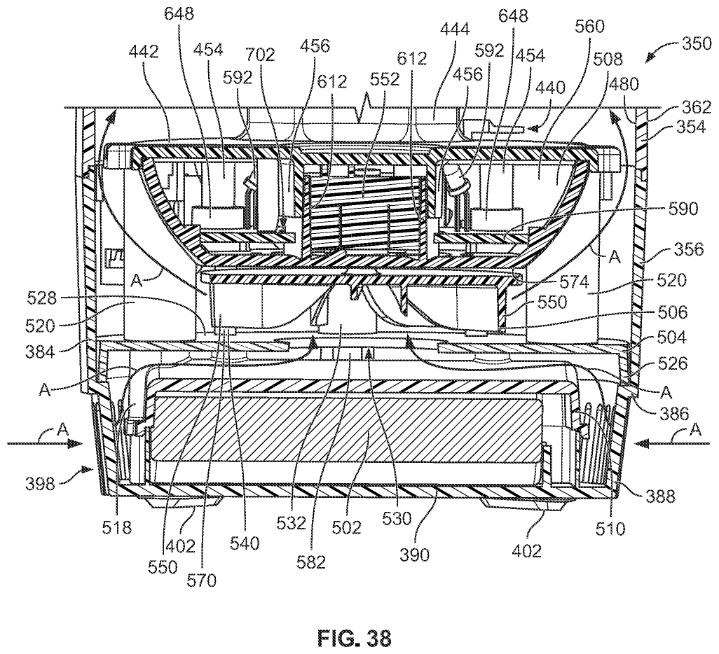

Referring still to FIG. 35, the air isolator 504 comprises an air isolator sidewall 526 and a planar surface 528. An intake aperture 530 extends through a center of the planar surface 528 of the air isolator 504. As illustrated in FIG. 35, the air isolator 504 comprises a generally circular shape with some interruptions adjacent the button 410. The air isolator 504 is positioned between the fan 506 and the battery 502. In preferred embodiments, the air isolator 504 is shaped to follow the general shape of the base 356. The air isolator 504 further comprises a plurality of air isolator posts 532 that are coupled with a plurality of sealed engine posts 540 by a plurality of fasteners 542 (see FIG. 36). Once assembled, the air isolator sidewall 526 may be positioned on the step 386 of the base 356 and adjacent the sidewall 384 of the base 356 (see FIG. 38). As will be discussed in further detail herein, the air isolator 504 helps to funnel the air through the base 356 via the intake aperture 530 to the fan 506.

Referring still to FIG. 35, the fan 506 is positioned directly above the intake aperture 530 of the air isolator 504 in order to draw air therethrough. The fan 506 includes a plurality of fan blades 550 and a motor 552 that is positioned within the sealed engine 508 (see FIG. 37). As will be discussed in further detail below, the fan 506 is configured to draw air through the plurality of vents 398 and out the chimney 380. In some embodiments, the fan 506 can have a fan speed of 3100-3600 rpm. Further, in some embodiments, the air flow associated with the fan 506 can be about 1.0 CFM. As noted herein, any type of fan can be used in the dispenser 350. In preferred embodiments, the fan 506 may be a centrifugal fan. Thus, the fan 506 can draw air in through the intake aperture 530 of the air isolator 504 and out around the sealed engine 508 and the stand assembly 440.

Referring still to FIG. 35, the sealed engine 508 is positioned on top of the fan 506 and underneath the platform 442. As will be discussed in further detail herein, the sealed engine 508 is configured to hold various electronics within the dispenser 350. Specifically, the sealed engine 508 is configured to protect the various electronics from water damage or other external elements. Therefore, in preferred embodiments, the sealed engine 508 is waterproof and/or dust proof. As illustrated in FIG. 35, the sealed engine 508 is bowl shaped, i.e., convex when looking up at the sealed engine 508, and comprises a sidewall 560. The sidewall 560 of the sealed engine 508 comprises a plurality of sealing flanges 562 and retention protrusions 564 extending therefrom. Each of the sealing flanges 562 comprises a sealing slot 566 that is configured to receive one of the plurality of hooks 452 of the platform 442 therethrough to secure the sealed engine 508 to the platform 442. The retention protrusions 564 extend outwardly from the sidewall 560 of the sealed engine 508, which operate to engage the shroud 354 when it is coupled with the base 356. The retention protrusions 564 are a type of retention mechanism that couples the shroud 354 with the base 356. Alternative retention mechanisms may include elements that cause a snap fit, a friction fit, or an interference fit. The sealed engine 508 can also be secured to the platform 442 by the plurality of platform posts 454 (see FIG. 37).

As discussed above, the sealed engine 508 can also comprise the plurality of sealed engine posts 540 that extend downward from the sidewall 560 of the sealed engine 508. In some embodiments, the plurality of sealed engine posts 540 can comprise a plurality of main posts 568 and a plurality of auxiliary posts 570, which are smaller than the plurality of main posts 568. As noted herein, the plurality of sealed engine posts 540 can comprise various shapes and sizes. As illustrated in FIG. 35, the sealed engine 508 also comprises a bottom wall 574 that is adjacent the fan 506. The fan 506 can extend through the bottom wall 574 of the sealed engine 508 to connect with the motor 552 (see FIG. 36). As noted herein, the platform 442 defines a top surface of the sealed engine 508.

Referring now to the cross-sectional views of FIGS. 36 and 37, internal components of the dispenser 350 are shown in greater detail. Referring to FIG. 36, the sealed engine 508 is shown being connected with the stand assembly 440 via the platform 442. Further, the sealed engine 508 is secured to the shroud 354 by the retention protrusions 564. In particular, the shroud 354 comprises a plurality of retention features 580 that extend inwardly from the underside 480 of the shroud 354. The retention protrusions 564 are configured to slide into the retention features 580 in order to secure the shroud 354 to the base 356, i.e., sealed engine 508. As illustrated in FIG. 36, the lower portion 388 of the base 356 further comprises a plurality of support posts 582 that help to further secure all of the interior components of the base 356 together. Specifically, each of the plurality of support posts 582 extend from the bottom wall 390 of the base 356 and into one of the air isolator posts 532. Each of the plurality of support posts 582 and the air isolator posts 532 are also connected/fastened with the sealed engine 508 via one of the fasteners 542. In other words, each of the plurality of main posts 568 of the sealed engine posts 540 is configured to mate with one of the air isolator posts 532, which includes one of the support posts 582 therein. Therefore, the air isolator posts 532, the main posts 568 of the sealed engine posts 540, and the support posts 582 are all coupled to each other by the plurality of fasteners 542. In the present embodiment, there are three fasteners 542 that secure the air isolator posts 532, the main posts 568 of the sealed engine posts 540, and the support posts 582 together, however, only a single fastener 542 is shown in the cross-section of FIG. 36.

Referring still to FIG. 36, a printed circuit board (PCB) 590 is shown within the sealed engine 508. A plurality of light emitting diodes (LEDs) 592 are shown electrically coupled with the PCB 590, the plurality of LEDs 592 being disposed above the PCB 590. In some embodiments, the plurality of LEDs 592 are disposed above and/or below the PCB 590. Further, in some embodiments, some of the LEDs 592 are disposed adjacent the front, rear, and sides of the dispenser 350. As noted herein, the LEDs 592 are intended to be used to emit light through the shroud 354 and/or the button 410 depending on the chosen setting, which can vary based on user preference. The LEDs 592 may alternatively be positioned in any suitable location within the dispenser 350. The one or more LEDs 592 may indicate that the dispenser 350 is on or off, may provide an alert, e.g., low battery, and/or may provide any other suitable indicator for a user. As further noted herein, a color and/or a brightness of the LEDs 592 may be adjusted depending on a desired brightness and/or color of light to be emitted through the shroud 354.

For example, the LEDs 592 may illuminate a first color or a first flashing color when the dispenser 350 is on, a second color or a second flashing color when the battery 502 is low, a third color or a third flashing color when the dispenser 350 is plugged into a power source, and additional colors depending on the setting of the dispenser 350. In some embodiments, the LEDs 592 may illuminate a dimed color or light depending on the settings. The dispenser 350 may also include one or more separate openings in the shroud 354 and/or base 356 to permit passage of the light emitted by each LED 592. Further, as discussed above, the dispenser 350 may include translucent or transparent portions to permit passage of the light emitted by each LED 592.

Referring still to FIG. 36, the button 410 is shown protruding through the base 356 and adjacent a switch 602. The switch 602 being operable to be adjusted between various settings of the dispenser 350. The switch 602 may be a push button switch that, when depressed, may cause the dispenser 350 to initiate one or more functions, such as turning the dispenser 350 on or off, causing an adjustment in the amount of fragrance that is dispensed, adjusting one or more settings on the dispenser 350, or adjusting the color or brightness of one or more of the LEDs 592. As illustrated in FIG. 36, the switch 602 may be electrically coupled with the PCB 590.

While the dispenser 350 is disclosed as having a particular switch, one skilled in the art will appreciate the dispenser 350 may include any number of switches and/or may include any suitable types of switches, for example, timing switches, on/off switches, setting switches, switches controlling another component within the assembly, such as a heater or the fan 506, and/or any other suitable switches.

Referring still to FIG. 36, the motor 552 is shown within the sealed engine 508. As discussed above, the motor 552 is configured to power and move the fan 506 during use. As noted herein, the motor 552 can comprise any type of motor in order to sufficiently work the fan 506. As discussed above, once assembled, the sealed engine 508 can provide protection to the motor 552, the LEDs 592, the PCB 590, and any other electronical components therein from external elements, i.e., water or dust. As illustrated in FIG. 36, the sealed engine 508 can comprise a plurality of support ribs 612 that interact with the plurality of platform ribs 456 to secure the motor 552 within the sealed engine 508 (see also FIG. 38). In some embodiments, the motor 552 can be received between the plurality of support ribs 612 and the plurality of platform ribs 456. The plurality of support ribs 612 are configured to surround and hold the motor 552 in place.

Referring still to FIG. 36, the stand assembly 440 is shown in cross section, and the components that are disposed therein are visible. In particular, the first and second wires 226 are shown extending from the PCB 590, through the stand 444, and into the manifold 450. As discussed above with respect to the dispenser 50, the first and second wires 226 of the dispenser 350 are electrically connected to the PCB 590 and to the piezoelectric assembly 110, which is disposed within the crown 448 of the stand assembly 440. In some embodiments, the first and second wires 226 may be positioned within a wire tube. As illustrated in FIG. 36, the stand 444 is fixedly coupled with the chassis 446, which is also fixedly coupled with the crown 448. The stand 444, the chassis 446, and the crown 448 are all separate components, and may be coupled with one another via a snap fit, friction fit, interference fit, adhesive, or another method of coupling. In some embodiments, the stand 444 may be attached to the chassis 446 and the crown 448 by a plurality of fasteners. As noted above, the stand 444, the chassis 446, and the crown 448 may all comprise polypropylene, or another polymeric material. As further illustrated in FIG. 36, the piezoelectric assembly 110 is centrally located along the longitudinal axis 80. While the stand 444, the chassis 446, the platform 442, the base 356, and the shroud 354 are illustrated as comprising a polymer, other types of materials are contemplated, such as glass, ceramic, wood, metal, etc.

Referring still to FIG. 36, a spring 620 is shown, which is used to apply a force against the piezoelectric assembly 110. The spring 620 applies a constant force against the piezoelectric assembly 110 to retain it in a static configuration until the refill 352 is positioned within the chassis 446 by a user. Similar to the spring 232 described above with respect to the dispenser 50, when a user inserts the refill 352 into the dispenser 350, the piezoelectric assembly 110 is translated upward, causing the spring 620 to compress. The spring 620 defines a spring wire 622 that has a wire diameter, which is further discussed below. The piezoelectric assembly 110 is disposed above a refill cavity 624, and is configured to receive the distal end 204 of the wick 188 when the refill 352 is inserted into the chassis 446 (see FIG. 41). As illustrated in FIG. 36, the chassis includes a piezo platform 630, which is unitary with the chassis 446 and defines a surface to which the piezoelectric assembly 110 is secured. Thus, the piezo platform 630 retains the piezoelectric assembly 110 in place, and prevents the piezoelectric assembly 110 from being displaced farther than the piezo platform 630 when the refill 352 is not secured to the chassis 446. The piezo platform 630 is generally circular and includes an aperture 632 in a center thereof, which allows the distal end 204 of the wick 188 to engage in physical contact with the piezoelectric assembly 110 when inserted into the chassis 446 (see FIG. 41). When the refill 352 is not inserted into the dispenser 350, the piezoelectric assembly 110 is positioned on the piezo platform 630 by way of the spring 620 forcing it thereon. Once the refill 352 is inserted into the dispenser 350, the piezoelectric assembly 110 will lift off the piezo platform 630 to accommodate the wick 188 of the refill 352 (see FIG. 41).

Referring still to FIG. 36, the chassis 446 includes a plurality of ledges 640 (only one ledge 640 illustrated in FIG. 36) that are positioned above the tabs 150. In other words, a single ledge 640 is positioned above each tab 150. The plurality of ledges 640 are configured to engage with a plurality of flanges 642 on the crown 448 (only one flange illustrated in FIG. 36). The engagement of the plurality of ledges 640 and the plurality of flanges 642 secure the chassis 446 and the crown 448 together. The chassis 446 and the crown 448 are snap fit together, however, the chassis 446 and the crown 448 may be coupled together in another fashion, such as via adhesive, fasteners, an interference fit, or a friction fit.

Referring now to FIG. 37, another cross-sectional view of the dispenser 350 is shown. The battery cover post 518 is illustrated extending into the sealed engine support 520 and the platform posts 454 are illustrated extending into cylindrical sleeves 648 of the sealed engine 508. Further, the refill guide ribs 230 are configured to snugly receive the refill 352 within the chassis 446. As illustrated in FIG. 37, the spring 620 comprises a top end 654 and a bottom end 656. The piezoelectric assembly 110 is also shown in FIG. 37, and is disposed below the bottom end 656 of the spring 620. The bottom end 656 of the spring 620 may be fixedly attached with the piezoelectric assembly 110, or may be separate but disposed in physical contact with one another. Therefore, the bottom end 656 of the spring 620 is formed to receive the piezoelectric assembly 110, which is in turn formed to receive the upper wick 192 when the refill 352 is engaged therewith.

Referring to FIGS. 36 and 37, the annular wall 490 defines a plurality of fingers 660 that extend downward from the top wall 472 of the crown 448 around the longitudinal axis 80. As noted herein, the plurality of fingers 660 are an extension of the annular wall 490 and are configured about the longitudinal axis 80. In other words, the annular wall 490 defines a plurality of openings 662 between the plurality of fingers 660. As illustrated in FIGS. 36 and 37, the top end 654 of the spring 620 is wrapped around the plurality of fingers 660 of the annular wall 490 and is in contact with the crown 448. Put differently, the top end 654 of the spring 620 is positioned around the plurality of fingers 660 of the annular wall 490. In some embodiments, the spring 620 can be secured to the annular wall 490 of the crown 448.

Referring still to FIGS. 36 and 37, the air deflector 476 is secured with the shroud 354 and the crown 448. The air deflector 476 comprises a frustoconical shape and includes an air wall 670 that extends around the crown 448. The air wall 670 is angled with respect to the longitudinal axis 80 and is in contact with the underside 480 of the shroud 354. Specifically, the air wall 670 is in contact with the spline-shaped lower portion 368 of the shroud 354 at a first end 672 and the frustoconical upper portion 370 of the shroud 354 at a second end 674. The second end 674 of the air wall 670 is positioned within a shroud groove 676 on the underside 480 of the shroud 354. The air wall 670 also comprises an air wall ledge 678 that is configured to receive a portion of the underside 480 of the shroud 354 (see FIG. 36). As illustrated in FIGS. 36 and 37, the underside 480 of the shroud 354 comprises a plurality of shroud posts 686 that extend through air deflector posts 688. The plurality of shroud posts 686 help further secure the air deflector 476 to the shroud 354. Further, the air deflector 476 is in contact with the plurality of ribs 474 on the crown 448. The plurality of ribs 474 allow the air deflector 476 to be spaced from the crown 448 and define a plurality of air channels 690 therebetween. As noted herein, the plurality of air channels 690 are defined by the air wall 670 of the air deflector 476 and the side surface 470 of the crown 448. During use, air is configured to travel through the plurality of air channels 690 via the fan 506 and dispense the fragrance from the refill 352. In other words, the air deflector 476 is configured to direct air out of the chimney 380. As noted herein, the air deflector 476 is coaxial with the chimney 380 and is positioned around the longitudinal axis 80. The air deflector 476 allows for air flow around the chimney 380 to be optimized and directed away from the dispenser 350. Further, the air deflector 476 helps to reduce air turbulence, condensation, and particle fallout around the dispenser 350. As discussed above, the plurality of supports 478 on the plurality of ribs 474 can contact the underside 480 of the shroud 354. Specifically, the supports 478 can contact the underside 480 of the shroud 354 between the lip 372 and the shroud groove 676.

Referring now to FIG. 38, a zoomed in cross-sectional view of the base 356 is shown with a plurality of arrows A illustrating the air flow through the base 356. While the fan 506 is running, air can move into the base 356 via the plurality of vents 398 and flow through the intake aperture 530 of the air isolator 504. After flowing through the intake aperture 530, the fan 506 can push the air around the sealed engine 508 and the platform 442. Referring now to FIG. 37, once the air has flowed around the platform 442 (see arrows A in FIG. 37), the air can flow up and around the stand assembly 440, through the air channels 690 between the air deflector 476 and the crown 448, and out of the chimney 380. As noted herein, in some embodiments, air may flow through the dispenser 350 even while the fan 506 is not on.

Referring now to FIG. 39, the refill 352 is shown in greater detail. As noted herein, the refill 352 is substantially similar to the refill 52 described above with respect to the dispenser 50. The main differences between the refill 352 and the refill 52 relate to the position of the annular rim 152 and the length of the finish 182 and the head 176. In particular, the refill 352 comprises a longer finish 182 and head 176 than the refill 52. Further, as a result of this longer finish 182, the position of the annular rim 152 of the refill 352 is lower, i.e., farther from the threading 154, than the refill 52. Therefore, the refill 352 comprises a larger height, i.e., taken parallel to the longitudinal axis 80, than the refill 52. In some embodiments, the plug 186 of the refill 352 may comprise a plurality of teeth 694 that secure the wick 188 in position. As noted herein, the refill 352 can also comprise the cap 146 (see FIG. 10). In some embodiments, the refill 352 may comprise a plurality of logos or labels thereon. Further, in some embodiments, the refill 352 may be formed from plastic, such as Polyethylene Terephthalate (PET). Still further, in some embodiments, the plastic may be a 100% Post-Consumer Recycled (PCR) plastic with a UV inhibitor. However, it is contemplated that the refill 352 can be formed from any type of plastic or polymer. As further noted herein, the refill 352 comprises the wick 188 (including the upper wick 192 and lower wick 194), as discussed above with respect to the refill 52.

In some embodiments, the refill 352 may come in a refill kit. For example, multiple refills 352 may be packaged and sold together to a user. Therefore, once the refill 352 runs out of volatile material within the dispenser 350, the refill 352 can be replaced with one of the refills within the refill kit. In some embodiments, the refill kit may include a box or container with 1, 2, 3, 4, 5, 6, 7, 8, 9, 10, or more refills 352 therein. Each of the refills 352 within the refill kit may comprise a different (or the same) fragrance or volatile material therein.

As noted herein, the volatile material disposed in the container 170 of the refill 352 may be any type of volatile material adapted to be dispensed into an environment. For example, the material within the container 170 may include a cleaner, an insecticide, an insect repellant, an insect attractant, a disinfectant, a mold or mildew inhibitor, a fragrance, a disinfectant, an air purifier, an aromatherapy scent, an antiseptic, an odor eliminator, a positive fragrancing volatile material, an air-freshener, a deodorizer, or the like, and combinations thereof. Additives may be included in the volatile material, such as, for example, fragrances and/or preservatives. In fact, any fluid may be provided within the container 170.

Referring now to FIG. 40, the piezoelectric assembly 110, the port assembly 412, the motor 552, the fan 506, the button 410, and the printed circuit board (PCB) 590 are shown in an isometric view. In other words, the electrical components within the sealed engine 508 are shown with the motor 552, the fan 506, the port assembly 412, the button 410, the wires 226, and the piezoelectric assembly 110. The PCB 590 comprises a generally annular shape with various notches therein. Further, the PCB 590 comprises a PCB aperture 702 that extends through the middle of the PCB 590. The motor 552 is configured to be positioned within and extend through the PCB aperture 702. As discussed above, the wires 226 are configured to connect with the PCB 590 and the piezoelectric assembly 110. Further, the switch 602 of the button 410 is connected with the PCB 590. In some embodiments, the motor 552 may be coupled or electrically coupled with the PCB 590. As illustrated in FIG. 40, the port assembly 412 comprises a port printed circuit board (PCB) 706 secured to the port 414. In some embodiments, the port PCB 706 may be electrically coupled with the PCB 590.

As noted herein, in some embodiments, the PCB 590 may comprise one or more controllers and/or one or more timers secured thereon and/ thereto, similar to the PCB 216 described above. Further, in some embodiments, a controller may be disposed within or along the PCB 590. Alternatively, in some embodiments, the dispenser 350 may comprise a controller that is a separate component that is electrically coupled with the PCB 590. Further, in some embodiments, the timer may be disposed within or along the PCB 590, or may be a separate component. In some embodiments, the wires 226 may be attached with a terminal (not shown) that is secured to the PCB 590. As noted herein, other electrical components, such as resistors, transistors, capacitors, processors, controllers, and relays may further be disposed along or within the PCB 590.

The LEDs 592, the button 410, the switch 602, and the wires 226 are shown clearly in the view of FIG. 40. In some embodiments, the dispenser 350 may comprise a limit switch that provides a signal to a controller when the shroud 354 is secured with the base 356, thereby not allowing the dispenser 350 to activate until the shroud 354 has been secured to the base 356. As illustrated in FIG. 40, four LEDs 592 are shown coupled with the PCB 590. In some embodiments, the dispenser 350 may comprises 1, 2, 3, 4, 5, 6, 7, 8, 9, or more LEDs 592. The different LEDs 592 may emit different colored lights, or may emit the same color. Further, the LEDs 592 may have the same intensity or a different intensity from one another. As noted herein, the PCB 590 can comprise any shape or profile that allows it to be securely retained with the profile of the base 356.

Referring still to FIG. 40, the piezoelectric assembly 110 is shown secured with the wires 226. As noted herein, the piezoelectric assembly 110 of the dispenser 350 is substantially identical to the piezoelectric assembly 110 described above with respect to the dispenser 50. Therefore, the piezoelectric assembly 110 of the dispenser 350 also comprises the piezoelectric plate 260 and the piezoelectric element 112 that circumscribes the periphery of the upper surface 262 of the piezoelectric plate 260. Further, the piezoelectric plate 260 further includes the central dome 264 that is generally concave and circular in shape. The central dome 264 comprises the apertured section 266 including the plurality of orifices 268, as discussed above with respect to the dispenser 50.

As noted herein, the piezoelectric assembly 110 of the dispenser 350 functions in the same way as the piezoelectric assembly 110 of the dispenser 50. Therefore, when the piezoelectric element 112 is energized, the piezoelectric plate 260 is caused to expand and contract, thereby releasing a volatile into the surrounding environment. While a particular piezoelectric element 112 is described, any actuator may be utilized, for example, a piezoelectric vibrating mesh actuator, a piezoelectric standing wave actuator, a piezoelectric vibrating needle, or any other suitable piezoelectric actuator. As further noted herein, the plurality of orifices 268 are positioned in a circular manner and define the array 269 (see FIG. 18B). Further, as noted herein, the wick 188 of the refill 352 can comprise the wick diameter DW (see FIG. 12) as discussed above and the plurality of orifices 268 of the dispenser 350 can define the array diameter DA (see FIG. 18) as discussed above.

As further discussed above, in some embodiments, the array 269 has a diameter or at least one width dimension of between about 0.5 mm and about 10.0 mm, or between about 1.0 mm and about 9.0 mm, or between about 2.0 mm and about 8.0 mm, or between about 3.0 mm and about 7.0 mm, or between about 3.0 mm and about 4.0 mm, or between about 3.5 mm and about 4.0 mm, or between about 3.7 mm and about 3.9 mm, or about 3.8 mm. As further discussed above, the wick diameter DW (see FIG. 12) and the array diameter DA (see FIG. 18) may define a ratio of DW/DA of between about 1.0 and about 3.0. In some embodiments, the ratio is about 1.1. In some embodiments, the ratio of DW/DA is between about 1.4 and about 1.5, or about 1.4, or about 1.5. In some embodiments, the wick diameter DW is between about 3.0 mm and about 5.5 mm, or between about 3.5 mm and about 4.0 mm, or about 3.8 mm. In some embodiments, the wick diameter DW is between about 5 mm and about 6 mm, or between about 5.2 mm and about 5.7 mm, or about 5.5 mm. In some embodiments, the array diameter DA is between about 3.0 mm and about 6.0 mm, or between about 4.0 mm and about 5.0 mm, or about 4.5 mm. In some embodiments, the array diameter DA is between about 3 mm and about 4.5 mm, or between about 3 mm and about 4 mm, or between about 3.5 mm and about 4 mm, or about 3.8 mm. The ratio of wick diameters may affect the accuracy and consistency of the plume of the volatile that is emitted by the dispenser 350. For example, having a ratio DW/DA of about 1.1 (or greater than 1.0) has been found to create a repeatable and accurate dispersal of volatile from the dispenser 350. In some embodiments the array 269 may comprise between about 100 and about 400 orifices, or between about 150 and about 350 orifices, or about 316 orifices, or about 200 orifices, or about 150 orifices.

Referring now to FIG. 41, a detail view of the upper end of the stand assembly 440 is shown with the refill 352 having been inserted. In some embodiments, a soft material, such as loading foam, may be provided along an underside of the piezoelectric assembly 110. As discussed above, additional materials may be provided along an underside of the piezoelectric assembly 110 to aid in accuracy or consistency of the plume of volatile generated when the dispenser 350 is activated.

Referring still to FIG. 41, the top end 654 of the spring 620 is shown wrapped around a plurality of fingers 660 of the annular wall 490, and the bottom end 656 of the spring 620 is shown in contact with and applying a force against the piezoelectric assembly 110. In other words, the top end 654 of the spring 620 surrounds the plurality of fingers 660 of the annular wall 490 and the bottom end 656 of the spring 620 contacts the piezoelectric element 112. As discussed above, the spring 620 is positioned to provide an opposing force against the refill 352 when it has been inserted into the chassis 446. Therefore, when the wick 188 is engaged with the piezoelectric assembly 110, the spring 620 is compressed and the piezoelectric assembly 110 is displaced a distance X (see FIG. 20) above the piezo platform 630.

Referring still to FIG. 41, the refill 352 is shown having been inserted into the manifold 450. Because the refill 352 has been inserted into the refill cavity 624 of the chassis 446, the spring 620 has been compressed, thus, the spring 620 is shown in a compressed configuration in FIG. 41. Still referring to FIG. 41, the rim 152 of the refill 352 is also shown engaged with the catches 236 of the tabs 150, and the distal end 204 of the wick 188 is shown in physical contact with the lower surface 276 of the piezoelectric assembly 110. As discussed above with respect to the dispenser 50, the compression of the spring 620 shown in FIG. 41 is a result of the size and type of spring 620 that is used in the dispenser 350, which may be chosen based upon a variety of factors. Other than the spring 620 having been compressed, the piezoelectric assembly 110 having been translated upward due to the force applied by the wick 188, and the catches 236 of the tabs 150 having been engaged with the rim 152 of the refill 352, no other elements of the manifold 450 are moved or manipulated when the refill 352 is inserted into the manifold 450. As noted herein, FIG. 41 shows the refill 352 in an operational state, where the dispenser 350 could be activated to disperse the volatile by providing an electrical pulse to the piezoelectric assembly 110. As discussed in detail above, an absorbent material (not shown) may be included between the wick 188 and the piezoelectric assembly 110.

During operation, the piezoelectric assembly 110 is actuated, either continuously or intermittently, to dispense volatile material. More particularly, as discussed above, an oscillating electric field is applied to the piezoelectric element 112, which causes expansion and contraction of the piezoelectric plate 260 in a radial direction. The expansion and contraction cause the piezoelectric plate 260 to vibrate in an axial direction (along a longitudinal axis 80 of the dispenser 350), forcing volatile material retained within the orifices of the piezoelectric plate 260 away from the piezoelectric assembly 110, through the passageway 492 defined by the annular wall 490, and through the chimney 380 of the shroud 354 (see arrow P defining plume of volatile). While the plume of volatile makes its way through the chimney 380, air being forced through the dispenser 350 by way of the fan 506 moves through the plurality of air channels 690 defined by the air deflector 476 (see arrows A defining flow of air via the fan 506). Therefore, air (see arrows A) will be flowing through the chimney 380 via the plurality of air channels 690 around the plume of volatile (see arrow P) while the plume of volatile makes its way out of the passageway 492 of the annular wall 490. In some embodiments, airflow (see arrows A) may completely surround the plume of volatile (see arrow P) leaving the passageway 492 of the annular wall 490. The airflow from the fan 506 (see arrows A) helps dispersion of the plume of the volatile (see arrow P). In other words, the airflow produced by the fan 506 (see arrows A) helps to disperse the plume of volatile (see arrow P) farther away from the dispenser 350, thereby allowing additional area to be covered by the plume of volatile. In some embodiments, the fan 506 allows for the plume of volatile to reach a greater area around the dispenser 350. In some embodiments, the dispenser 350 may not comprise the fan 506.

Referring still to FIG. 41, the spring wire 622 of the spring 620 may comprise a diameter, i.e., wire diameter, of 0.55 mm. In some embodiments, the spring 620 may include an uncompressed height, i.e., taken in a direction parallel to the longitudinal axis 80, of between about 5.0 mm and about 12.0 mm, or about 8.0 mm. In some embodiments, the spring 620 may have between about 2 turns and about 10 turns, or about 5.5 turns. In some embodiments, the top end 654 of the spring 620 may comprise a coil diameter, i.e., entire spring diameter taken along a line orthogonal to the longitudinal axis 80, that is smaller than the coil diameter at the bottom end 656 of the spring 620. In some embodiments, the top end 654 of the spring 620 may comprise a coil diameter of about 12.9 mm and the bottom end 656 of the spring 620 may comprise a coil diameter of about 16.0 mm. Further, in some embodiments, the top end 654 and the bottom end 656 of the spring 620 may comprise the same coil diameter (see FIG. 41). In some embodiments, the spring 620 has an uncompressed height of about 8.0 mm. Furthermore, in some embodiments, the spring 620, when compressed to about 5.0 mm long, i.e., taken in a direction parallel to the longitudinal axis 80, has a force of about 28 gf (0.3 N).

Referring to FIGS. 32, 36, 38, 39, and 41, a method of inserting the refill 352 into the stand assembly 440 and assembling the dispenser 350 are disclosed. Specifically, the refill 352 is positioned with the head 176 thereof being inserted into the manifold 450, similar to the position illustrated in FIG. 23. The refill 352 is then moved upward so that the head 176 is inserted into the manifold 450. Once the rim 152 of the refill 352 has been engaged with the catches 236 of the tabs 150, the refill 352 can be characterized as being secured within the manifold (see FIG. 41). In some embodiments, the cap 146 (see FIG.10) may be inserted into the well 460 of the platform 442 to securely retain the cap 146 while the refill 352 is in use. Once the refill 352 is secured to the chassis 446, the shroud 354 can be inserted over the stand assembly 440. Specifically, the shroud 354 is aligned with the base 356 and is then rotated until the retention protrusions 564 of the base 356 engage with the retention features 580 along the underside 480 of the shroud 354 (see FIG. 36). Therefore, as the shroud 354 is rotated, the retention protrusions 564 of the base 356 slide into the retention features 580 of the shroud 354 to secure the shroud 354 to the base 356.

Referring back to FIG. 26, the button 410 can be configured to turn on the dispenser 350 and toggle between different settings. For example, if the button 410 is pressed for longer than 1 second, the dispenser 350 can power on, i.e., turn on the piezoelectric assembly 110, LEDs, and the fan 506. In some embodiments, once the dispenser 350 is on, if the button 410 is pressed again for less than 1 second, the dispenser 350 may change settings. For example, the lights may change color or brightness, the fan 506 may increase or decrease its speed, the fragrance strength may change, and/or the duty cycle of the piezoelectric assembly 110 and/or the fan 506 may change. As noted herein, the duty cycle of the piezoelectric assembly 110 and the fan 506 defines the intervals that the piezoelectric assembly 110 and the fan 506 are on and off. In preferred embodiments, the duty cycle for the piezoelectric assembly 110 and the fan 506 is 7 seconds on and 30 seconds off. Therefore, the piezoelectric assembly 110 and the fan 506 will be on for 7 seconds and then off for 30 seconds before coming back on for an additional 7 seconds and so forth. It is contemplated that the piezoelectric assembly 110 and the fan 506 may comprise any type of duty cycle. Further, depending on the settings of the dispenser 350, the dispenser 350 may be able to change the duty cycle times for the piezoelectric assembly 110 and the fan 506.

As noted herein, the dispenser 350 is configured to be used in an outdoor environment. Therefore, as discussed above, the dispenser 350 is water and dust resistant. In particular, the sealed engine 508 can be waterproof once it is sealed with the platform 442. Thus, the electronic components within the sealed engine 508, i.e., the PCB 590, the LEDs 592, and the motor 552, can be protected from rain or other external outdoor elements. In some embodiments, a cap (not shown) can be placed over the piezoelectric assembly 110 and/or the chimney 380 when the device is not in use to further protect these elements from the environment. Further, in some embodiments, the plurality of vents 398 in the base 356 can also provide water drainage through the dispenser 350. Furthermore, since the dispenser 350 is configured to be used for/provide outdoor protection, the LEDs 592 can also offer sufficient outdoor lighting to a user.

In some embodiments, the dispenser 350 may incorporate IoT (Internet of Things) capabilities. For example, in some embodiments, the dispenser 350 may be capable of connecting to the internet, i.e., WIFI, and/or connect with a user device, i.e., smart phone, smart watch, tablet, etc. The user may then be able to customize the settings of the dispenser 350 via the user device. In some embodiments, the user may be able to turn on/off the dispenser 350, switch the settings or duty cycle of the dispenser 350, switch the color of the lights of the dispenser 350, and/or determine the amount of battery left on the dispenser 350 by way of the user device. In some embodiments, the dispenser 350 may be controlled on the user device through an app.

Any of the embodiments described herein may be modified to include any of the structures or methodologies disclosed in connection with other embodiments. Further, although directional terminology, such as front, back, upper, lower, vertical, horizontal, etc. may be used throughout the present specification, it should be understood that such terms are not limiting and are only utilized herein to convey the orientation of different elements with respect to one another.

### INDUSTRIAL APPLICABILITY

Dispensers are commonly used to dispense a variety of volatile materials such as air fresheners, deodorants, insecticides, germicides, perfumes, and the like, that are stored within refill containers. Piezoelectric engines allow the volatile materials to be volatilized and then distributed into an environment in order for the contents thereof to be released without human interaction, for example, continuously or according to a predetermined time schedule.

We claim:

1. A volatile material dispenser, comprising:
a base including a fan and a battery therein;
a stand assembly that is coupled with the base, the stand assembly including a platform, a stand that extends from the platform, and a manifold that extends from the stand;
a shroud positioned on the base and defining a chimney; and
an air deflector positioned between the shroud and the manifold,
wherein the air deflector is configured to direct air out of the chimney.

2. The volatile material dispenser of claim 1, wherein the air deflector comprises a frustoconical shape and includes an air wall.

3. The volatile material dispenser of claim 2, wherein a plurality of air channels are defined by the air wall of the air deflector and a side surface of the manifold.

4. The volatile material dispenser of claim 2, wherein the air wall extends around the manifold.

5. The volatile material dispenser of claim 1, wherein the base further includes a plurality of vents.

6. The volatile material dispenser of claim 5, wherein the base further includes an air isolator having an intake aperture.

7. The volatile material dispenser of claim 6, wherein the air isolator is positioned between the fan and the battery.

8. The volatile material dispenser of claim 6, wherein, during use, air is configured to flow into the plurality of vents, through the intake aperture of the air isolator, and out of the chimney.

9. A volatile material dispenser, comprising:
a base including a fan and a battery therein, the base defining a lower portion that comprises a plurality of vents;
a stand assembly that is coupled with the base, the stand assembly including a manifold, the manifold containing a piezoelectric assembly;
a shroud defining a chimney; and
an air deflector positioned within the shroud,
wherein, during use, air is configured to flow through the plurality of vents and out of the chimney.

10. The volatile material dispenser of claim 9, wherein the base further comprises an air isolator and a sealed engine.

11. The volatile material dispenser of claim 10, wherein the sealed engine is waterproof

11. The volatile material dispenser of claim 10, wherein the sealed engine is positioned above the fan and underneath a platform of the stand assembly.

12. The volatile material dispenser of claim 10, wherein a printed circuit board and a plurality of light emitting diodes are positioned within the sealed engine.

13. The volatile material dispenser of claim 12, wherein the piezoelectric assembly is connected with the printed circuit board.

14. The volatile material dispenser of claim 10, wherein the fan comprises a motor that is positioned within the sealed engine.

15. The volatile material dispenser of claim 9, wherein the volatile material dispenser is configured to be used in an outdoor environment.

16. A volatile material dispenser, comprising:
a base;
a stand assembly that is coupled with the base, the stand assembly including a manifold, the manifold containing a piezoelectric assembly;
a fan;
a refill having a wick, the refill being removably coupled with the manifold and comprising a volatile material therein; and
a shroud positioned around the manifold and the refill, the shroud defining a chimney,
wherein the manifold includes an annular wall, and the annular wall defines a passageway,
wherein the annular wall is coaxial with the chimney and the piezoelectric assembly, and
wherein, during use, a plume of volatile is configured to flow through the passageway and the chimney by way of the piezoelectric assembly.

17. The volatile material dispenser of claim 16, wherein the wick of the refill is in contact with the piezoelectric assembly when the refill is coupled with the manifold.

18. The volatile material dispenser of claim 16, wherein the volatile material dispenser further comprises an air deflector that is secured with the shroud and the manifold.

19. The volatile material dispenser of claim 18, wherein the air deflector is configured to direct air out of the chimney and around the plume of volatile.

20. The volatile material dispenser of claim 16, wherein the base further includes a plurality of vents and an air isolator.
